(19) United States
(12) Patent Application Publication
(10) Pub. No.: US 2025/0001409 A1
(43) Pub. Date: Jan. 2, 2025

(54) DEVICES FOR DETECTION OF NEUTROPHIL LEVEL

(71) Applicant: 52 NORTH HEALTH LTD, Cambridge (GB)

(72) Inventors: Nicole Weckman, Cambridge (GB); Ama Frimpong, Cambridge (GB); Alexandra Meehan, Cambridge (GB); Luke Semple, Cambridge (GB); Russell Farn, Cambridge (GB); Daniel Godfrey, Cambridge (GB); Adrian Walker, Cambridge (GB)

(21) Appl. No.: 18/697,402

(22) PCT Filed: Oct. 4, 2022
(86) PCT No.: PCT/EP2022/077588

§ 371 Date: Mar. 29, 2024

(30) Foreign Application Priority Data
Oct. 4, 2021 (GB) 2114182.5

(51) Int. Cl. B01L 3/00

(52) U.S. Cl. CPC B01L 3/5023; B01L 3/502753; B01L 2300/0681; B01L 2300/069; B01L 2300/0803

(57) ABSTRACT

An assay device configured to separate a suspension into a filtrate and a residue for testing at least one of the filtrate and the residue. The device comprises a filter aperture for a filter membrane for receiving the suspension and separating the suspension into the filtrate and the residue. The device further comprises a filtrate receiving portion; a residue receiving portion; a first buffer container; a liquid release member configured to create an outlet of the first buffer container; a filter arm comprising the filter aperture; and a user actuated switch comprising a transition structure configured to guide the filter arm. During a transition from a first set position to a second set position of the user actuated switch, the filter arm is moved such that the filter aperture is aligned with the residue receiving portion; and the liquid release member and the first buffer container are brought together.

### FIELD OF THE DISCLOSURE

The disclosure relates to the field of portable assay devices.

### BACKGROUND

It is known to measure properties of either the liquid or the solid part of a suspension for monitoring health. For example, a blood sample may be used either to measure levels of specific biomarkers in the plasma, or to measure neutrophil levels (part of a white blood cell count). The biomarkers in the plasma may be measured using a lateral flow test. Measurement of neutrophil levels may be conducted using centrifugation as described by Oh H., Siano B., Diamond S. (2008). Neutrophil Isolation Protocol. JoVE. 17. http://www.jove.com/index/Details.stp?ID=745, doi: 10.3791/745.

Conventionally, these known techniques require the action of a trained professional to prepare the sample and to then undertake the desired test. Measurement of both liquid and solid parts of the suspension would require two samples to be processed. Often, the sample is provided by a patient at to a medical centre with a significant time delay between the point in time when the sample is provided and when the sample is processed and results are available.

### SUMMARY OF THE DISCLOSURE

Against this background, there is provided an assay device configured to separate a suspension into a filtrate and a residue for testing at least one of the filtrate and the residue. The device comprises a filter aperture for a filter membrane for receiving the suspension and separating the suspension into the filtrate and the residue. The device further comprises a filtrate receiving portion for receiving the filtrate. The device further comprises a residue receiving portion for receiving the residue. The device further comprises a first buffer container configured to contain a first buffer liquid. The device further comprises a liquid release member configured to create an outlet of the first buffer container. The device further comprises a filter arm comprising the filter. The device further comprises a user actuated switch configured to be movable from a first set position to a second set position, the user actuated switch comprising a transition structure configured to guide the filter arm. The assay device is configured such that at the first set position the filter aperture is aligned with the filtrate receiving portion. The assay device is further configured such that at the second set position the filter aperture is aligned with the first buffer container and with the residue receiving portion. The assay device is further configured such that during a first transition from the first set position to the second set position the filter arm is moved such that the filter aperture is aligned with the residue receiving portion; and the liquid release member and the first buffer container are brought together such that the liquid release member creates the outlet of the first buffer container.

In this way, a user may separate a suspension into a filtrate and residue in order to test at least one of the filtrate and residue, by simple mechanical actuation of a user actuated switch.

### BRIEF DESCRIPTION OF THE DRAWINGS

A specific embodiment of the disclosure will now be described, by way of example only, with reference to the accompanying drawings in which:

FIG. 1 shows a schematic drawing of a perspective view of the assay device in accordance with an embodiment of the present disclosure.

FIG. 2 shows a schematic drawing of the transition structure of the assay device in accordance with an embodiment of the present disclosure.

FIG. 3 shows schematic drawings of the user actuated switch of the assay device in accordance with an embodiment of the present disclosure. FIG. 3A shows a perspective view of the upper side of the user actuated switch that is actuated by the user. FIG. 3B shows the lower side of the user actuated switch, comprising first, second and third buffer capsules.

FIG. 4 shows a schematic drawing of the filter arm in accordance with an embodiment of the present disclosure.

FIG. 5 shows a schematic drawing of the sample receiving arm in accordance with an embodiment of the present disclosure.

FIG. 6 shows schematic drawings of the housing of the assay device in accordance with an embodiment of the present disclosure. FIG. 6A shows the shows the outside of the top of the housing. FIG. 6B shows the inside of the top of the housing. FIG. 6C shows the inside of the base of the housing.

FIG. 7 shows schematic drawings of how various components of part of the assay device fit together in accordance with an embodiment of the present disclosure. FIG. 7A shows a side view of the base of the housing, the transition structure, the filter arm and the sample receiving arm. FIG. 7B shows a top view of the top of the housing and the user actuated switch.

FIG. 8 shows schematic drawings of how various components of part of the assay device fit together in accordance with an embodiment of the present disclosure. FIG. 8A shows a top view of the sample receiving arm, the transition structure and the base of the housing.

FIG. 8B shows the filter arm, the sample receiving arm, the transition structure and the base of the housing.

FIG. 9 shows a schematic view of the sample receiving arm resting on the transition structure in accordance with an embodiment of the present disclosure. FIG. 9A shows a perspective view of the base of the housing, the sample receiving arm and the transition structure. FIG. 9B shows a magnified section of FIG. 9A.

FIG. 10 shows a schematic drawing of how the filter arm is positioned relative to the sample receiving arm and the transition structure, in accordance with an embodiment of the present disclosure. FIG. 10A shows a perspective view of the base of the housing, the sample receiving arm, the filter arm and the transition structure. FIG. 10B shows a magnified section of FIG. 10A, showing the filter arm and sample receiving arm resting on the transition structure. FIG. 10C shows a magnified section of FIG. 10A, showing the filter arm attached to the sample receiving arm.

FIG. 11 shows a graph of the level of the transition structure on which the filter arm and sample receiving arm rest for each set position, in accordance with an embodiment of the present disclosure.

FIG. 12 shows schematic drawings of top views of parts of the assay device in the third set position, in accordance with an embodiment of the present disclosure. FIG. 12A shows the top of the housing and the user actuated switch. FIG. 12B shows the filter arm, the sample receiving arm, the user actuated switch and the base of the housing. FIG. 12C shows the filter arm, the sample receiving arm, the transition structure and the base of the housing.

FIG. 13 shows schematic drawings of perspective views of parts of the assay device in the third set position, in accordance with an embodiment of the present disclosure. FIG. 13A shows the user actuated switch, the filter arm, the sample receiving arm and the base of the housing. FIG. 13B shows the filter arm, the sample receiving arm, the transition structure and the base of the housing. Filter 13C shows a magnified section of FIG. 13B, showing the filter aperture aligned with the filtrate receiving portion.

FIG. 14 shows schematic drawings of top views of parts of the assay device in the first set position, in accordance with an embodiment of the present disclosure. FIG. 14A shows the top of the housing and the user actuated switch. FIG. 14B shows the filter arm, the sample receiving arm, the transition structure and the base of the housing.

FIG. 15 shows schematic drawings of top views of parts of the assay device in the second set position, in accordance with an embodiment of the present disclosure. FIG. 15A shows the top of the housing and the user actuated switch. FIG. 15B shows the filter arm, the sample receiving arm, the transition structure and the base of the housing.

FIG. 16 shows a schematic drawing of a side view of parts of the assay device the transition from the second set position in accordance with an embodiment of the present disclosure, showing the filter arm, the sample receiving arm, the transition structure and the base of the housing.

FIG. 17 shows schematic drawings of top views of parts of the assay device in the fourth set position, in accordance with an embodiment of the present disclosure. FIG. 17A shows the top of the housing and the user actuated switch. FIG. 17B shows the filter arm, the sample receiving arm, the transition structure and the base of the housing.

FIG. 18 shows a schematic drawing a side view of parts of the assay device in the fourth set position, in accordance with an embodiment of the present disclosure.

FIG. 19 shows schematic drawings of perspective views of parts of the assay device in the fourth set position, in accordance with an embodiment of the present disclosure. FIG. 19A shows a perspective view of the filter arm, the sample receiving arm, the transition structure and the base of the housing. FIG. 19B shows a magnified section of FIG. 19A, showing the filter arm and sample receiving arm resting on the transition structure.

FIG. 20 shows a graph of the level of the transition structure on which the filter arm and sample receiving arm rest at the third, first, second, and fourth set positions and during the transitions between the set positions, in accordance with an embodiment of the present disclosure.

FIG. 21 shows a schematic cross section of the filter arm, a buffer pod and the user actuated switch in accordance with an embodiment of the present disclosure.

FIG. 22 shows a schematic diagram of an assay device in accordance with an embodiment of the present disclosure. FIG. 22A shows a top view, FIG. 22B shows a perspective view, FIG. 22C shows a side view and FIG. 22D shows an end view.

FIG. 23 shows a schematic diagram of a dial of an assay device in accordance with an embodiment of the present disclosure. FIG. 23A shows a top view, FIG. 23B shows a cross-section and FIG. 23C shows a perspective view from below.

FIG. 24 shows a schematic diagram of a filter arm of an assay device in accordance with an embodiment of the present disclosure. FIG. 24A shows the filter arm arranged with a dial, FIG. 24B shows a top view and FIG. 24C shows a perspective view from above.

FIG. 25 shows a schematic diagram of an assay device in accordance with an embodiment of the present disclosure in the third set position. FIG. 25A shows a top view. FIG. 25B shows a top view, with the dial and buffer cassette removed to show the filter arm. FIG. 25C shows a top view, with the dial removed to show the buffer cassette. FIG. 25D shows a perspective view, with the dial and an upper portion of the housing removed to show the buffer cassette.

FIG. 26 shows a schematic diagram of an assay device in accordance with an embodiment of the present disclosure during a transition from the third set position to the first set position. FIG. 26A shows a top view. FIG. 26B shows a top view, with the dial removed to show the buffer cassette. FIG. 26C shows a top view, with the dial and buffer cassette removed to show the filter arm.

FIG. 27 shows a schematic diagram of an assay device in accordance with an embodiment of the present disclosure in the first set position. FIG. 27A shows a top view. FIG. 27B shows a perspective view, with an upper portion of the housing removed. FIG. 27C shows a top view, with the dial removed to show the buffer cassette. FIG. 27D shows a perspective view, with the dial and an upper portion of the housing removed to show the buffer cassette. FIG. 27E shows a top view with the dial and buffer cassette removed to show the filter arm. FIG. 27F shows a perspective view with the dial and buffer cassette removed to show the filter arm.

FIG. 28 shows a schematic diagram of an assay device in accordance with an embodiment of the present disclosure in the second set position. FIG. 28A shows a top view. FIG. 28B shows a top view, with the dial and buffer cassette removed to show the filter arm. FIG. 28C shows a top view, with the dial removed to show the buffer cassette. FIG. 28D shows a perspective view, with the dial and an upper portion of the housing removed to show the buffer cassette.

FIG. 29 shows a schematic diagram of an assay device in accordance with an embodiment of the present disclosure in the first set position. FIG. 29A shows a top view. FIG. 29B shows a cross-section taken along line D-D.

FIG. 30 shows a schematic diagram of an assay device in accordance with an embodiment of the present disclosure during the transition from the third set position to the first set position. FIG. 30A shows an end view, and FIG. 30B shows a cross-section taken along line P-P.

FIG. 31 shows a schematic diagram of an assay device in accordance with an embodiment of the present disclosure in the first set position, with the dial and an upper portion of the housing removed to show the buffer cassette, and with a magnified portion.

FIG. 32 shows a schematic diagram of an assay device in accordance with an embodiment of the present disclosure, with the dial, buffer cassette, filter arm and an upper portion of the housing removed, and showing a ramp. FIG. 32A shows a perspective view with a magnified portion. FIG. 32B shows a side view. FIG. 32C shows a cross-section taken along line L-L, with a magnified portion.

FIG. 33 shows a schematic diagram of an assay device in accordance with an embodiment of the present disclosure in the second set position. FIG. 33A shows a top view, with an upper portion of the housing removed. FIG. 33B shows a cross-section taken along the line T-T.

### DETAILED DESCRIPTION

### Assay Device According to Embodiments of the Present Disclosure

An assay device according to an embodiment of the present disclosure is configured to separate a suspension into a filtrate and a residue, for testing at least one of the filtrate and the residue.

The assay device comprises a filter aperture for a filter membrane, wherein the filter membrane is used to separate the suspension into the filtrate and the residue. The assay device further comprises a filtrate receiving portion for receiving the filtrate that has passed through the filter aperture. The assay device also comprises a residue receiving portion that may receive the residue that is left on the filter membrane. The residue receiving portion may receive the residue still on the filter membrane, or the residue may be transferred from the filter membrane to the residue receiving portion. The residue may undergo further processing prior to being transferred to the residue receiving portion. A filter arm comprises the filter aperture.

The assay device comprises a first buffer container (also referred to as a processing buffer container) configured to contain a first buffer liquid (also referred to as a processing buffer liquid). A liquid release member is configured to create an outlet in the first buffer container such that the first buffer liquid is released and passes through the filter aperture.

The assay device further comprises a user actuated switch configured to be movable from a first set position to a second set position. The user actuated switch comprises a transition structure configured to guide the filter arm. At the first set position the filter aperture is aligned with the filtrate receiving portion. At the second set position the filter aperture is aligned with the first buffer container and with the residue receiving portion. During a first transition from the first set position to the second set position the filter arm is moved such that the filter aperture is aligned with the residue receiving portion, and the liquid release member and the first buffer container are brought together such that the liquid release member creates the outlet of the first buffer container.

In use, the assay device may begin with the user actuated switch in the first set position. The user deposits the suspension onto a filter membrane that is in the filter aperture. At the first set position, the filter aperture is aligned with the filtrate receiving portion such that when the suspension is deposited on the filter membrane, any filtrate that passes through the filter membrane is received by the filtrate receiving portion. The user moves the user actuated switch from the first set position to the second set position, and during this first transition the filter arm is moved such that the filter aperture is aligned with the residue receiving portion. The liquid release member and the first buffer container are brought together such that the liquid release member creates the outlet of the first buffer container. The first buffer liquid washes through the filter membrane onto the residue receiving portion. The first buffer liquid may process the residue such that a residue component can pass through the filter membrane. The first buffer liquid may also wash the residue component through the filter membrane.

In an embodiment, the filter arm may rest on the transition structure. Moving the user actuated switch moves the transition structure, which in turn guides the filter arm. In an embodiment, the transition structure may comprise a plurality of levels such that moving the transition structure guides the filter arm up or down between the plurality of levels.

In an embodiment, the assay device further comprises a second buffer container (also referred to as a filtering buffer container) configured to contain a second buffer liquid (also referred to as a filtering buffer liquid). The residue receiving portion is configured to receive the residue and the processing buffer liquid through the filter aperture. The filtrate receiving portion may be configured to receive the filtrate and the filtering buffer liquid through the filter aperture. The user actuated switch may be configured to be movable from a third set position to the first set position and from the first set position to the second set position. At the third set position the filter aperture may be aligned with the filtrate receiving portion. At the first set position the filter aperture may be aligned with the filtrate receiving portion and with the filtering buffer container. The liquid release member may be further configured to create an outlet of the filtering buffer container. In use, the assay device may begin with the user actuated switch in the third set position. The user deposits the suspension onto a filter membrane that is in the filter aperture. During a transition from the third set position to the first set position the liquid release member and the filtering buffer container may be brought together such that the liquid release member creates the outlet of the filtering buffer container. The filtering buffer liquid may pass through the filter aperture and be received by the filtrate receiving portion. At the second set position the filter aperture may be aligned with the residue receiving portion and with the processing buffer container. In a transition from the first set position to the second set position the filter arm is moved such that the filter aperture is aligned with the residue receiving portion, and the liquid release member and the processing buffer container are brought together such that the liquid release member creates the outlet of the processing buffer container.

The assay device may be configured such that the filtering buffer liquid washes the filtrate through the filter membrane and onto the filtrate receiving portion. The assay device may be configured such that the processing buffer may process the residue to form a residue component that may pass through the filter membrane. The assay device may be further configured such that the processing buffer washes the residue component through the filter membrane.

In an embodiment, the user actuated switch may be further configured to be movable to a fourth set position, wherein at the fourth set position the filter aperture is aligned with the residue receiving portion and not with the first buffer container (processing buffer container). The assay device may comprise a third buffer container (also referred to as the chasing buffer container) configured to contain a third buffer liquid (also referred to as the chasing buffer liquid), and wherein in the fourth set position the filter aperture is aligned with the residue receiving outlet and the chasing buffer container. The fourth set position may be after the second set position, such that the user actuated switch is configured to be movable from the first set position to the second set position, and from the second set position to the fourth set position. During the transition from the second set position to the fourth set position the liquid release member and the chasing buffer container may be brought together such that the liquid release member creates the outlet of the chasing buffer container. At the fourth set position, the chasing buffer liquid may pass through the filter aperture and be received by the residue receiving portion.

In an embodiment, the assay device may comprise the processing buffer container (wherein the user actuated switch is configured to be movable from the first set position to the second position). In an embodiment, the assay device may comprise the filtering buffer container and the processing buffer container (wherein the user actuated switch is configured to be movable from the third set position to the first set position and from the first set position to the second position). In an embodiment, the assay device may comprise the processing buffer container and the chasing buffer container (wherein the user actuated switch is configured to be movable from the first set position to the second position and from the second set position to the fourth set position). In an embodiment, the assay device may comprise the filtering buffer, the processing buffer container and the chasing buffer container (wherein the user actuated switch is configured to be movable from the third set position to the first set position, from the first set position to the second position and from the second set position to the fourth set position).

In an embodiment the user actuated switch may comprise a dial. The dial may be configured to be rotated from the first set position to the second set position. The dial may be configured to be rotated from the third set position to the first set position and from the first set position to the second set position. The dial may be configured to be rotated from the third set position to the first set position, from the first set position to the second set position, and from the second set position to the fourth set position. The dial may be configured to be rotated from the first set position to the second set position and from the second set position to the fourth set position. The transition structure may be configured to be rotated by rotating the dial.

In another embodiment the user actuated switch may comprise a slider, wherein the slider is configured to be moved from the first set position to the second set position. The slider may be configured to be moved from the third set position to the first set position and from the first set position to the second set position. The slider may be configured to be moved from the first set position to the second set position and from the second set position to the fourth set position. The slider may be configured to be moved from the third set position to the first set position, from the first set position to the second set position, and from the second set position to the fourth set position. The slider may be configured to be moved in one dimension. For example, the slider may be configured to move from the third set position to the first set position and in the same one dimension from the first set position to the second set position. Alternatively, the slider may be configured to be moved in a plurality of dimensions. For example, the slider may be configured to be movable in one dimension from the third set position to the first set position and in a different one dimension from the first set position to the second set position.

In embodiments, the transition structure may comprise a plurality of levels. The plurality of levels of the transition structure may be connected via a plurality of ramps. The transition structure may be configured to guide the filter arm along the plurality of levels and between the plurality of levels, in response to movement of the user actuated switch between the set positions. The filter arm further may comprise a first contact point configured to rest on the transition structure.

In an embodiment the plurality of levels may comprise concentric arcs. Each of the plurality of levels may comprise one or more arcs.

The assay device may further comprise a sample receiving arm comprising the filtrate receiving portion and the residue receiving portion. The transition structure may be configured to guide the sample receiving arm in response to movement of the user actuated switch. The sample receiving arm may further comprise a contact aperture and a second contact point configured to rest on the transition structure, wherein the filter arm rests on the sample receiving arm and wherein the first contact point is configured to pass through the contact aperture and rest on the transition structure. In this way, the sample receiving arm and filter arm may both be guided by the transition structure, but may rest on the same or different levels.

The plurality of levels comprise a first level, a second level below the first level and a third level below the second level. In an embodiment, during the transition from the third set position to the first set position the first contact point may move from the third level to the second level, and the second contact may move from the second level to the first level. In another embodiment, during the first transition the first contact point may move from the second level to the third level and then to the second level, and the second contact point may move from the first level to the second level and then to the first level. The liquid release member may create the outlet of the filtering buffer container when the first contact point moves from the third level to the second level.

In an embodiment, during the transition from the first set position to the second set position the first contact point may move from the second level to the third level, and the second contact point may move from the first level to the second level. The first contact point may then move from the third level to the second level and the second contact point may remain at the second level, such that the upper arm is lifted relative to the lower arm and the upper arm rotates relative to the lower arm such that the filter aperture is aligned with the residue receiving portion. The liquid release member may create the outlet of the processing buffer container when the first contact point moves from the third level to the second level.

In an embodiment, during the transition from the second set position to the fourth set position the first contact point may move from the second level to the third level and the second contact point may remain at the second level. The first contact point may then move from the third level to the second level and the second contact point may move from the second level to the first level. The liquid release member may create the outlet of the chasing buffer container when the first contact point moves from the third level to the second level.

In other embodiments, the transition structure may be configured to rotate the filter arm in response to movement of the user actuated switch between set positions. The user actuated switch may comprise a dial, wherein the dial and the filter arm rotate about the same axis. The assay device may comprise a filtering buffer container and a processing buffer container. In an example the filtering buffer container may be configured to contain a chase buffer and the processing buffer container may be configured to contain a lysis buffer. The dial may be configured to be rotated from the third set position to the first set position and from the first set position to the second set position. As described above, at the third set position the filter aperture may be aligned with the filtrate receiving portion. At the first set position the filter aperture may be aligned with the filtrate receiving portion, and the filtering buffer container may be aligned with the filtrate receiving portion. At the second set position the filter aperture may be aligned with the residue receiving portion and the processing buffer container may be aligned with the residue receiving portion.

In use, the assay device may begin with the user actuated switch in the third set position. The user deposits the suspension onto a filter membrane that is in the filter aperture. During a transition from the third set position to the first set position the filtering buffer container may be moved to be aligned with the filtrate receiving portion, the liquid release member and filtering buffer container may be brought together such that the liquid release member creates the outlet of the filtering buffer container. The filtering buffer liquid may pass through the filter aperture and be received by the filtrate receiving portion. During the transition from the third set position to the first set position the processing buffer container may be moved to be aligned with the residue receiving portion. At the second set position the filter aperture may be aligned with the residue receiving portion and with the processing buffer container. In a transition from the first set position to the second set position the filter arm may be moved such that the filter aperture is aligned with the residue receiving portion, and the liquid release member and the processing buffer container may be brought together such that the liquid release member creates the outlet of the processing buffer container.

The dial may be configured to rotate the filter buffer container and the processing buffer container about the same axis of rotation as the dial and the filter arm.

In an embodiment, at least one of the filtrate receiving portion and the residue receiving portion may comprise a lateral flow strip. In the event that the filtrate receiving portion comprises a lateral flow strip, the filtrate travels along the lateral flow strip. If present, the filtering buffer liquid washes the filtrate along the lateral flow strip. In the event that the residue receiving portion comprises a lateral flow strip, either the processing buffer liquid or the chasing buffer liquid washes along the lateral flow strip.

The filtrate receiving portion may comprise a test for the filtrate and the residue receiving portion may comprise a test for the residue. The filtrate receiving portion may direct the filtrate towards a test, and the residue receiving portion may direct the residue towards a test.

Each of the buffer liquids may comprise any liquid. For example, one or more of the buffer liquids may comprise a liquid configured to maintain a particular pH. One or more of the buffer liquids may comprise deionised water. The processing buffer liquid may comprise a lysing solution configured to lyse a component of a cell from the cell and then to wash the component into the residue receiving portion. The buffer liquids may comprise other liquids not mentioned in the examples provided here.

An additional liquid may be supplied externally. For example, the assay device may comprise a first buffer container configured to contain a first buffer liquid, wherein the assay device is configured to receive an additional buffer liquid externally. The additional liquid may be provided by the user, for example via a pipette. A user may add the first buffer liquid to the first buffer container.

The assay device may comprise a plurality of liquid release members. For example, the assay device may comprise a liquid release member for each buffer container.

### First Specific Embodiment

A first specific embodiment of the disclosure will now be described, by way of example. It will be understood from the preceding description that this disclosure covers both this first specific example, and other examples.

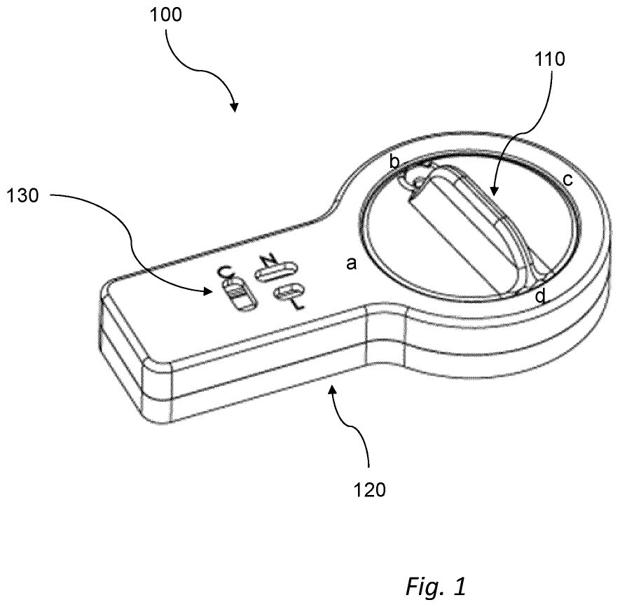

FIG. 1 shows a perspective view of a first specific embodiment of an assay device 100 in accordance with the disclosure. The assay device 100 comprises a user actuated switch comprising a dial 110. The assay device 100 comprises a housing 120. In the example shown in FIG. 1, the housing comprises apertures 130 for viewing results of testing the suspension. The assay device 100 of the specific embodiment comprises first, second and third buffer containers (not visible in FIG. 1). The user actuated switch is movable between first, second, third and fourth set positions. In FIG. 1, the third set position is marked as “a”, the first set position is marked as “b”, the second set position is marked as “c”, and the fourth set position is marked as “d”. The set positions may be marked in any appropriate way.

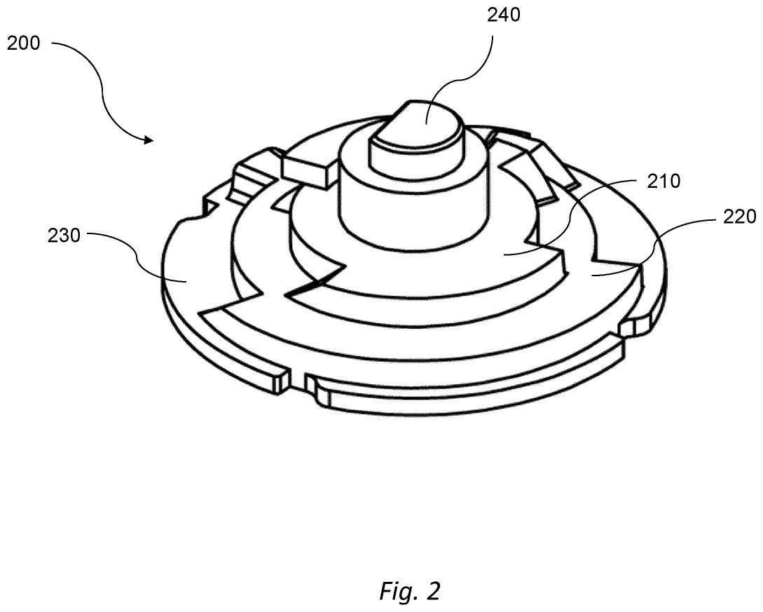

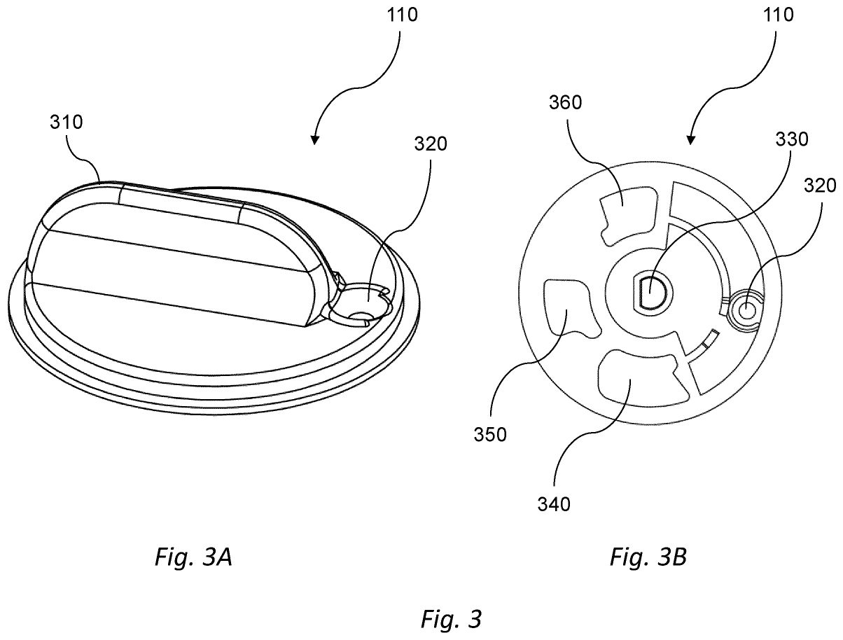

FIGS. 2 to 5 show schematic diagrams of individual components of the assay device 100 of the first specific embodiment. FIG. 2 shows transition structure 200. Transition structure 200 comprises a first level 210, a second level 220 and a third level 230. Each of the first, second and third levels comprises concentric arcs. Ramps between the first, second and third levels 210, 220 and 230 are configured to guide the filter arm between the levels. The transition structure 200 is connected to the dial 110 via connector 240. The connector 240 may, for example, be a D shape. The connector 240 may connect to a corresponding aperture (330, see FIG. 3) in the dial 110.

FIG. 3 shows the user actuated switch in this embodiment comprises the dial 110. FIG. 3A shows a perspective view of the top of the dial 110, as seen by the user. In use, the suspension is deposited into the sample receiving aperture 320 and the user turns the dial using the handle 310. The sample receiving aperture 320 is also used to show which of the set positions the dial 110 is in, by aligning with markings on the housing 120. FIG. 3B shows the bottom of the dial 110, which is positioned above the transition structure 200 on the interior of the assay device. The connector 240 of the transition structure 200 slots into aperture 330 of the dial 110. The underside of sample receiving aperture 320 is shown. The dial 110 comprises the second buffer container (filtering buffer container) 340, the first buffer container (processing buffer container) 350 and the third buffer container (chasing buffer container) 360.

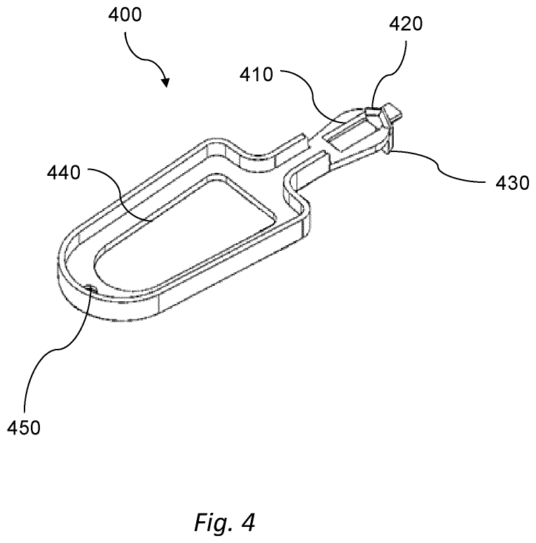

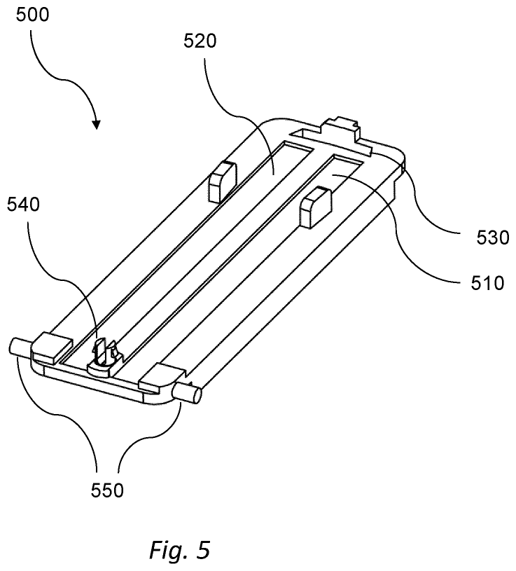

FIG. 4 shows a filter arm 400. The filter arm 400 comprises a filter aperture 410 for a filter membrane. The filter arm 400 further comprises a liquid release member 420 on the upper side of the filter arm 400 that is configured to create an opening in the filtering, processing and chasing buffer containers 340, 350 and 360. The filter arm 400 further comprises a first contact point 430 on the lower side of the filter arm. The viewing aperture 440 allows the sample receiving arm below the filter arm to be visible through apertures 130 in the housing 120. The filter arm 400 is connected to a sample receiving arm (500, see FIG. 5) via a connector aperture 450.

FIG. 5 shows a sample receiving arm 500 comprising a filtrate receiving portion 510 and a residue receiving portion 520. In this embodiment, the filtrate receiving portion 510 and the residue receiving portion 520 comprise lateral flow strips. The sample receiving arm 500 further comprises a contact aperture 530, wherein the first contact point 430 of the filter arm 400 is configured to pass through the contact aperture 530 to rest on the transition structure 200. The sample receiving arm 500 comprises a second contact point (not shown in FIG. 5, labelled as 560 in later figures) that is configured to rest on the transition structure. A connector 540 is configured to fit into connector aperture 450 of the filter arm 400, such that the sample receiving arm 500 connects rotatably to the filter arm 400. The connector 540 may, for example, comprise a snap pin with wings so that the filter arm 400 may rotate freely but not come loose perpendicular to the sample receiving arm 500. The sample arm 500 is configured to connect hingedly to the housing 120 via pins 550.

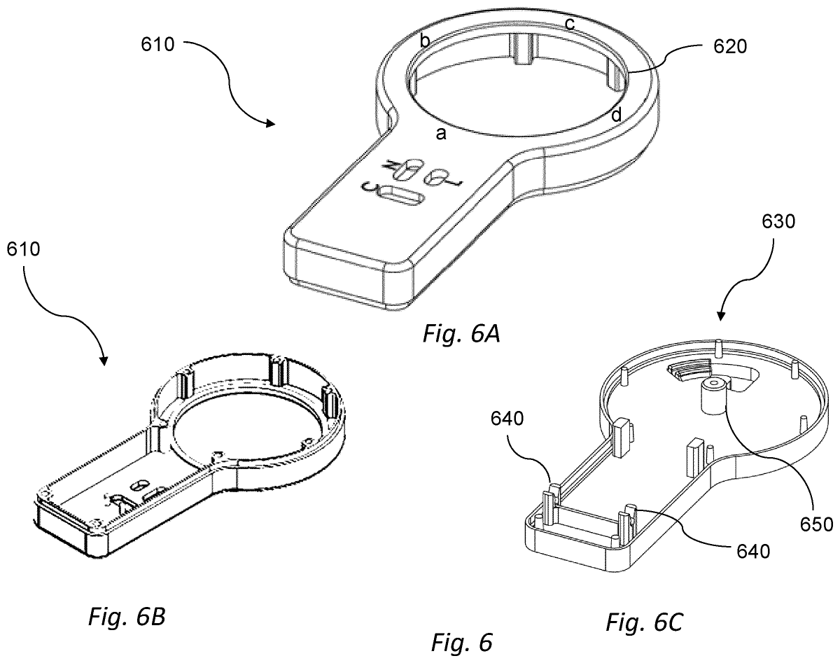

FIG. 6 shows the components of the housing 120. FIG. 6A shows the outer side of the top 610 of the housing 120, comprising an aperture 620 for the dial 110. FIG. 6B shows the inside of the top 610 of the housing 120. FIG. 6C shows the inside of the base 630 of the housing 120, comprising brackets 640 configured to hold the pins 550 of the sample receiving arm, and connector 650 configured to connect rotatably to the transition structure 200.

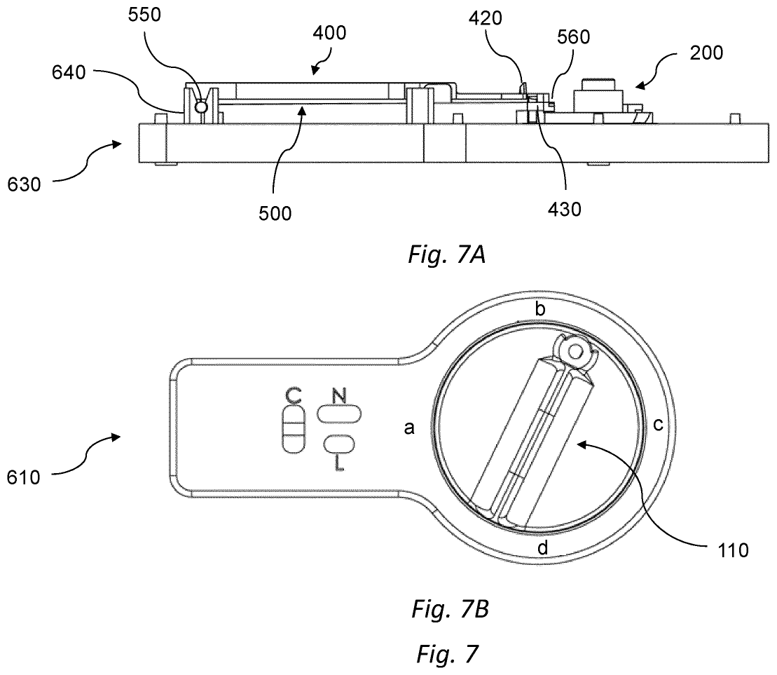

FIGS. 7 to 10 illustrate how various components of the assay device 100 fit together. FIG. 7A shows a side view of the base 630 of the housing 120, the transition structure 200, the filter arm 400 and the sample receiving arm 500 below the filter arm 400. The liquid release member 420, the first contact point 430, the second contact point 560, the pins 550 and brackets 640 are shown. First contact point 430 and second contact point 560 rest on the transition structure 200. FIG. 7B shows the top 610 of the housing 120, and the dial 110.

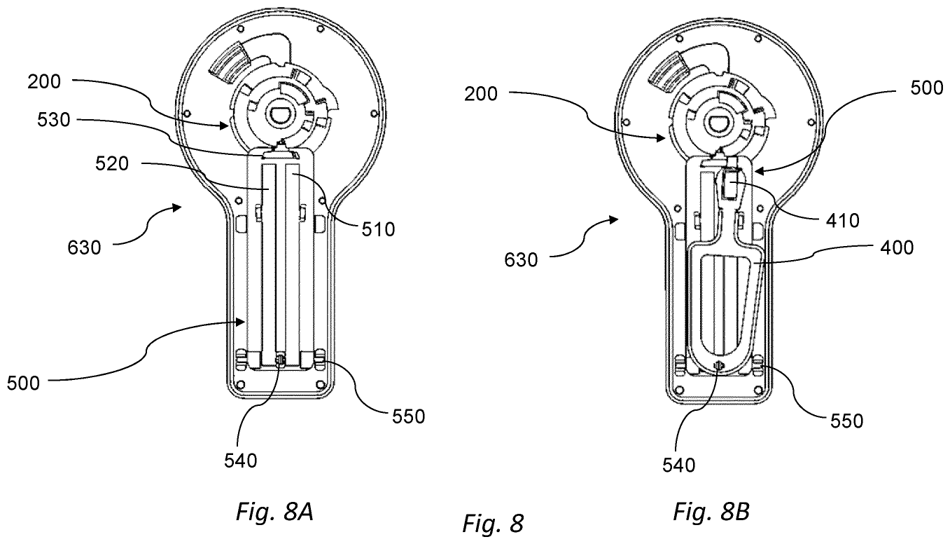

FIG. 8A shows a top view of the base 630 of the housing 120, with the transition structure 200 and the sample receiving arm 500 in situ. The filtrate receiving portion 510, the residue receiving portion 520, the contact aperture 530, the pins 550, and the connector 540 of the sample receiving arm 500 are shown. FIG. 8B shows a top view similar to that of FIG. 8A, with the addition of the filter arm 400 in situ above the sample receiving arm 500. Filter aperture 410 is visible in this view. The filter arm 400 is connected to the sample receiving arm 500 via the connector 540.

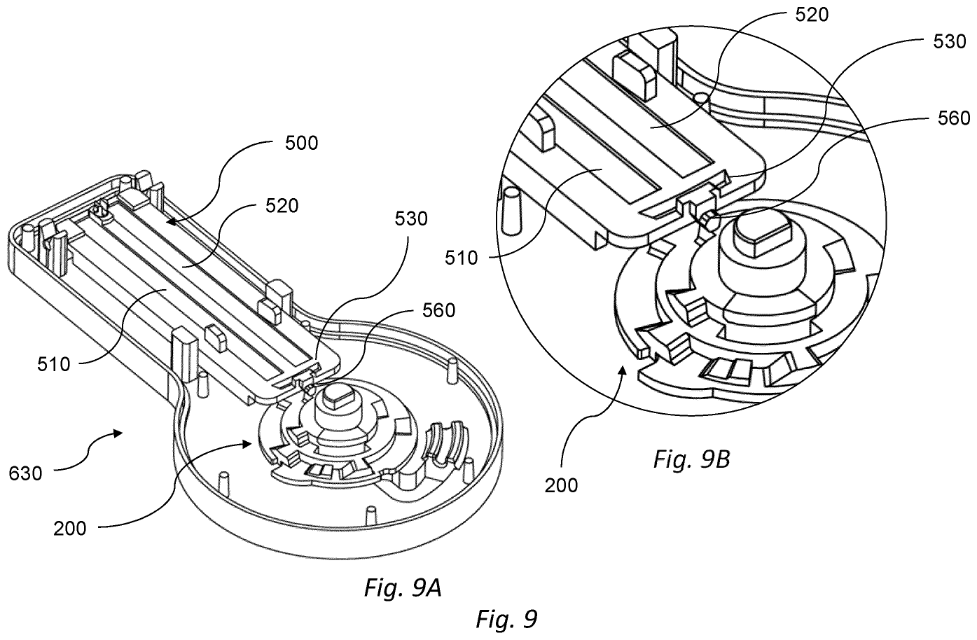

FIG. 9A shows a perspective view of the base of the housing 630, with the transition structure 200 and the sample receiving arm 500 in situ. FIG. 9B shows a magnified section of FIG. 9A, in which the second contact point 560 is shown resting on the transition structure 200.

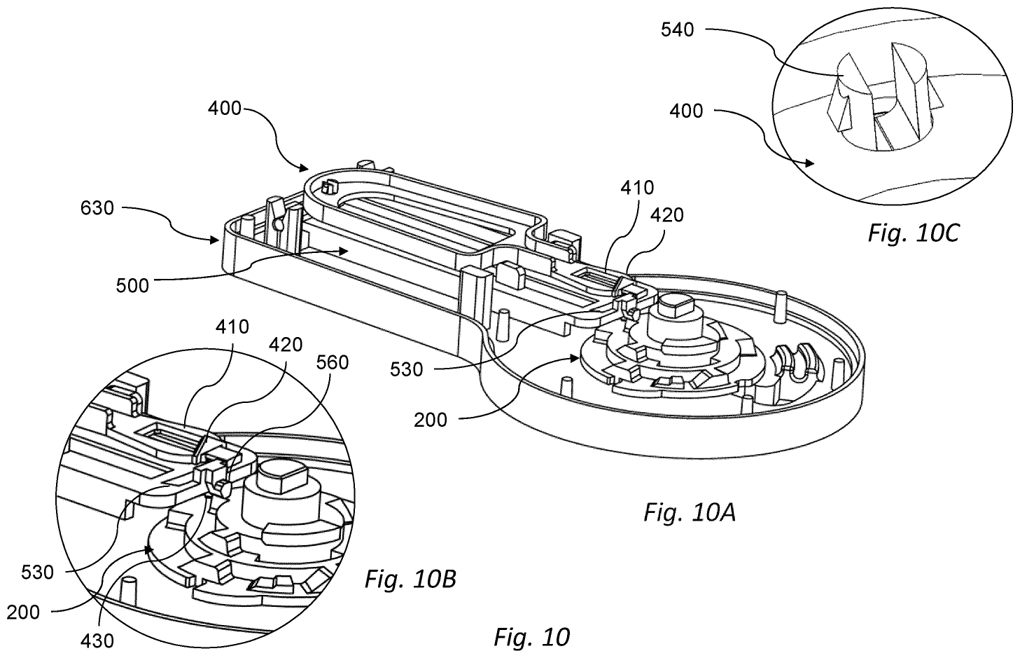

FIG. 10A shows a perspective view of the base 630 of the housing 120, with the transition structure 200, the filter arm 400 and the sample receiving arm 500 in situ. FIG. 10B shows a magnified section of FIG. 10A, in which the second contact point 560 is shown resting on the transition structure 200 and the filter aperture 410 is shows aligned with the residue receiving portion 520. The first contact point 430 passes through the contact aperture 530 to rest on the transition structure 200. FIG. 10C shows a magnified section of FIG. 10A, in which the connector 540 of the sample receiving arm 500 is shown connecting to the filter arm 400 via connector aperture 450.

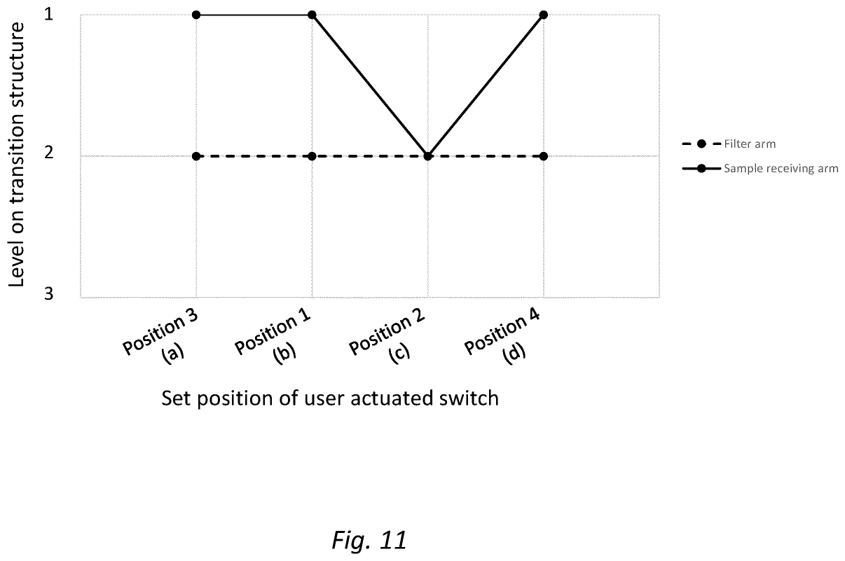

FIG. 11 shows a graph indicating which level of the transition structure 200 the first and second contact points 430 and 560 are resting on at each set position. At the each set position, the first contact point 430 (of the filter arm 400) rests on the second level 220. At the third, first and fourth set positions (a, b and d), the second contact point 560 (of the sample receiving arm 500) rests on the first level 210. At the second set position c (between first and fourth set positions), the first contact point 430 rests on the second level 220.

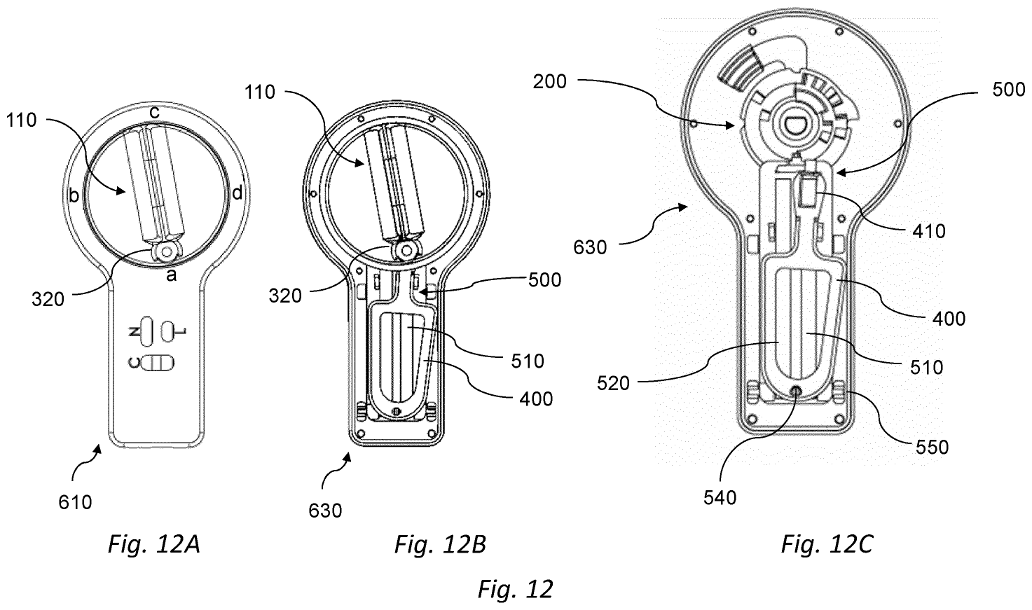

At the third set position (position a), the filter aperture 410 is aligned with the filtrate receiving portion 510, and the sample receiving aperture 320 is aligned with the filter aperture 410. In use, the user deposits the sample in the sample receiving aperture 320 and the sample passes onto the filter membrane held in the filter aperture 410. FIG. 12 shows top views of the assay device 100 in position a. FIG. 12A shows the top 610 of the housing 120, the dial 110 and the sample receiving aperture 320. FIG. 12B shows the assay device 100 with the top 610 of the housing 120 removed. FIG. 12B shows the base 630 of the housing 120, the sample receiving arm 500, the filter arm 400 and the dial 110. The filter aperture 410 is aligned with the filtrate receiving portion 510. FIG. 12C shows the assay device 100 with the dial 110 removed. FIG. 12C shows the base 630 of the housing 120, the sample receiving arm 500, the filter arm 400 and the transition structure 200. The filter aperture 410 is aligned with the filtrate receiving portion 510.

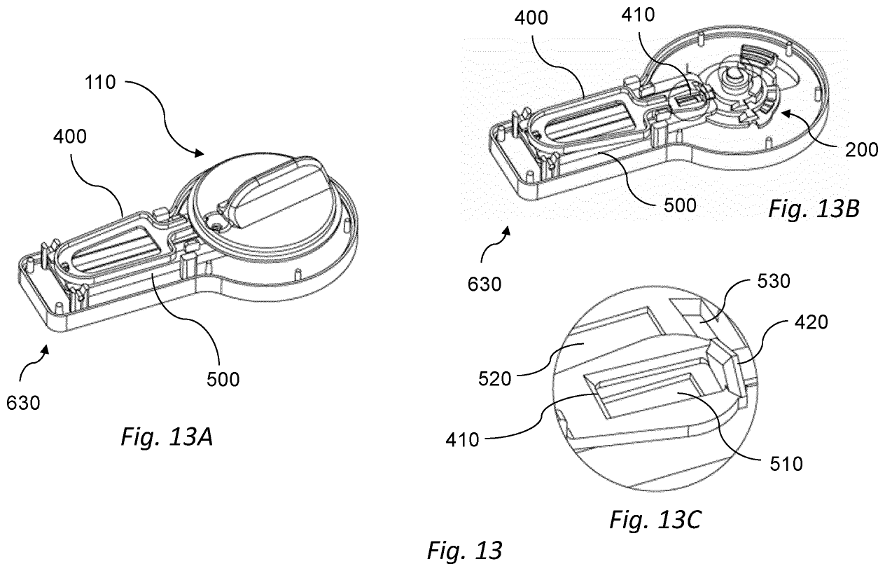

FIG. 13 shows perspective views of the assay device 100 in position a, with the filter aperture 410 and sample receiving aperture 320 both aligning with the filtrate receiving portion 510. FIG. 13A corresponds to the components shown in FIG. 12B. FIG. 13B corresponds to the components shown in FIG. 12C. FIG. 13C is a magnified section of FIG. 13B, showing the filter aperture 410 aligning with the filtrate receiving portion 510.

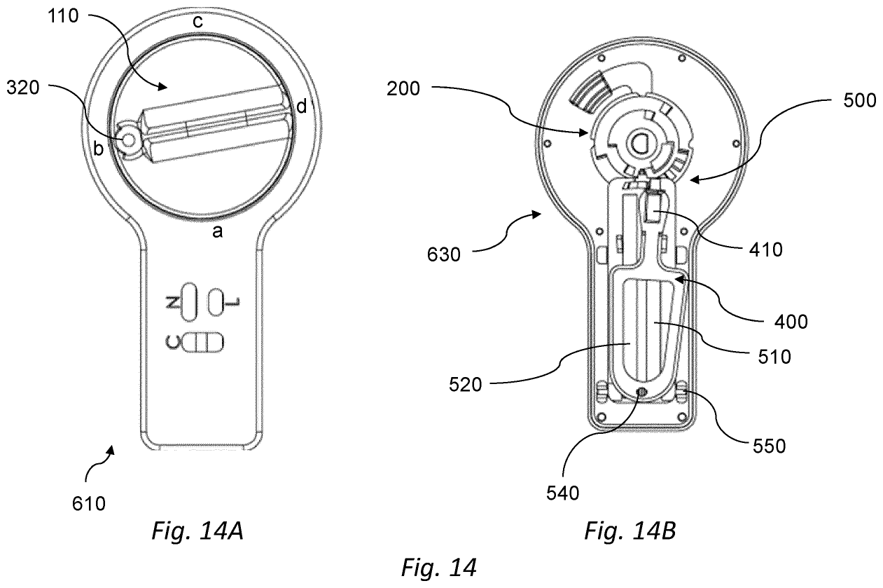

With reference to FIG. 14, at the first set position (position b) the filter aperture 410 is aligned with the filtrate receiving portion 510. FIG. 14A shows the top 610 of the housing 120, the dial 110 and the sample receiving aperture 320. FIG. 14B shows the assay device 100 with the top 610 of the housing 120 and the dial 110 removed. FIG. 14B shows the base 630 of the housing 120, the sample receiving arm 500, the filter arm 400 and the transition structure 200. The filter aperture 410 is aligned with the filtrate receiving portion 510.

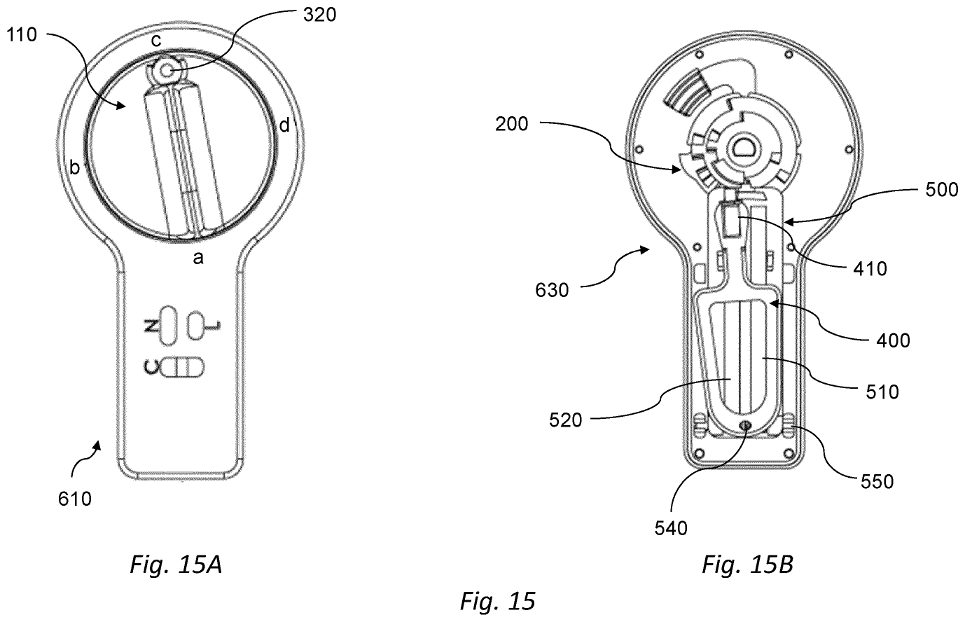

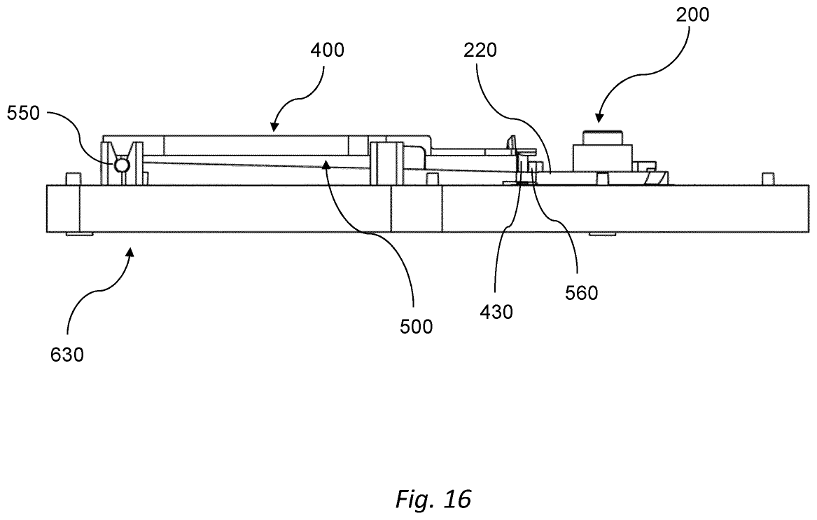

With reference to FIG. 15, at the second set position (position c) the filter aperture 410 is aligned with the residue receiving portion 520. FIG. 15A shows the top 610 of the housing 120, the dial 110 and the sample receiving aperture 320. FIG. 15B shows the assay device 100 with the top 610 of the housing 120 and the dial 110 removed. FIG. 15B shows the base 630 of the housing 120, the sample receiving arm 500, the filter arm 400 and the transition structure 200. The filter aperture 410 is aligned with the residue receiving portion 520. FIG. 16 shows a side view of the base 630 of the housing 120, the sample receiving arm 500, the filter arm 400 and the transition structure 200 in position c. Both the first contact point 430 and the second contact point 560 rest on the second level 220 of the transition structure.

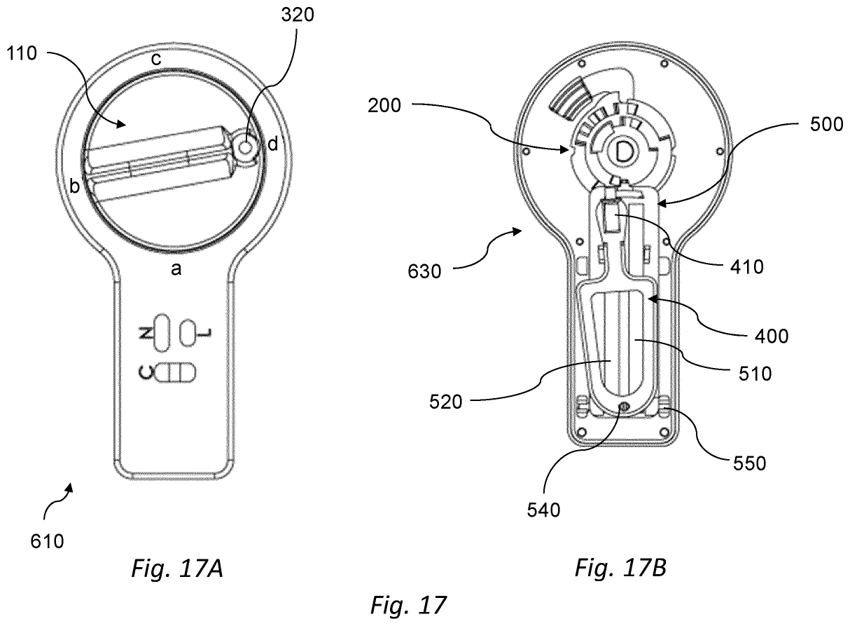

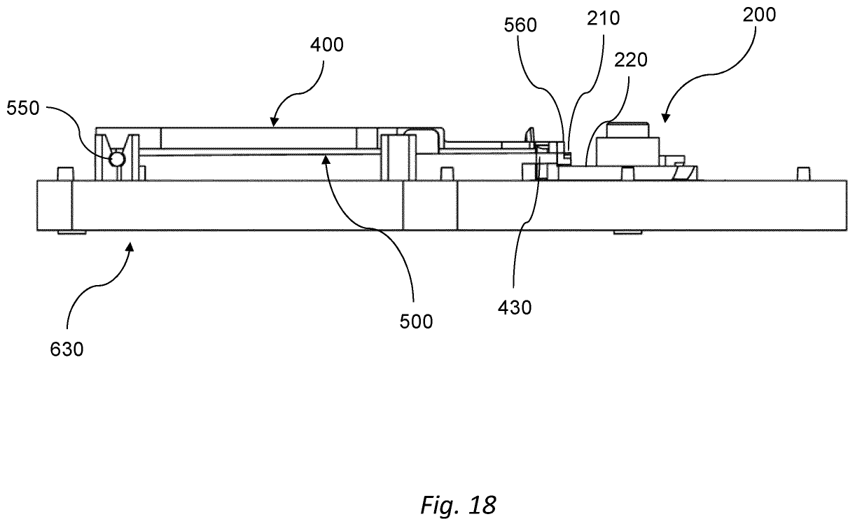

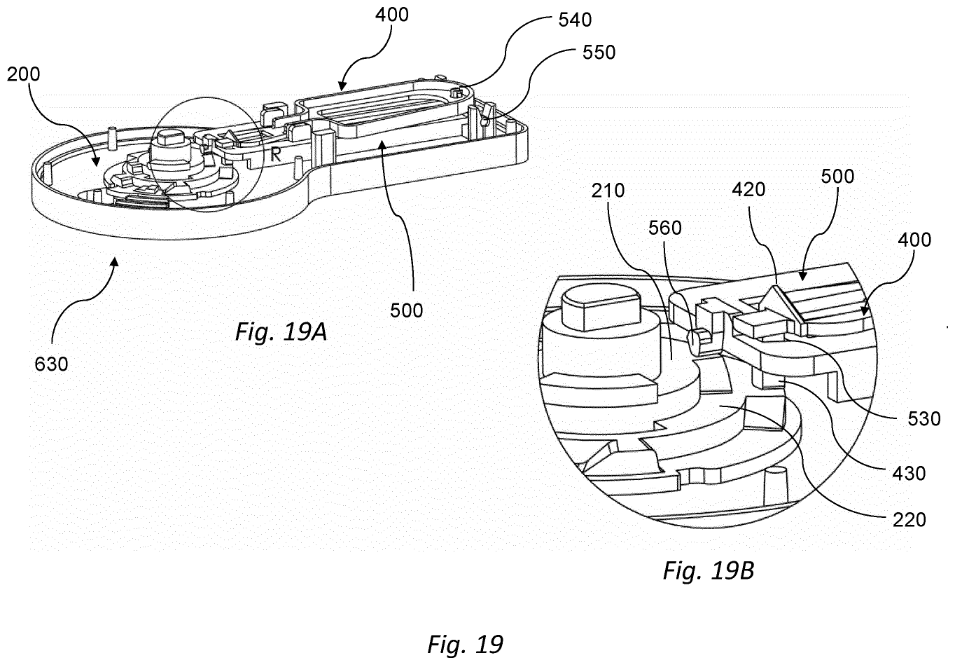

With reference to FIG. 17, at the fourth set position (position d) the filter aperture 410 is aligned with the residue receiving portion 520. FIG. 17A shows the top 610 of the housing 120, the dial 110 and the sample receiving aperture 320. FIG. 17B shows the assay device 100 with the top 610 of the housing 120 and the dial 110 removed. FIG. 17B shows the base 630 of the housing 120, the sample receiving arm 500, the filter arm 400 and the transition structure 200. The filter aperture 410 is aligned with the residue receiving portion 520. FIG. 18 shows a side view of the base 630 of the housing 120, the sample receiving arm 500, the filter arm 400 and the transition structure 200 in position d. The first contact point 430 rests on the second level 220 of the transition structure, and the second contact point 560 rests on the first level 210 of the transition structure. FIG. 19A shows a perspective view of the base 630 of the housing 120, the sample receiving arm 500, the filter arm 400 and the transition structure 200 in position d. FIG. 19B shows a magnified section of FIG. 19A. The first contact point 430 rests on the second level 220 of the transition structure, and the second contact point 560 rests on the first level 210 of the transition structure.

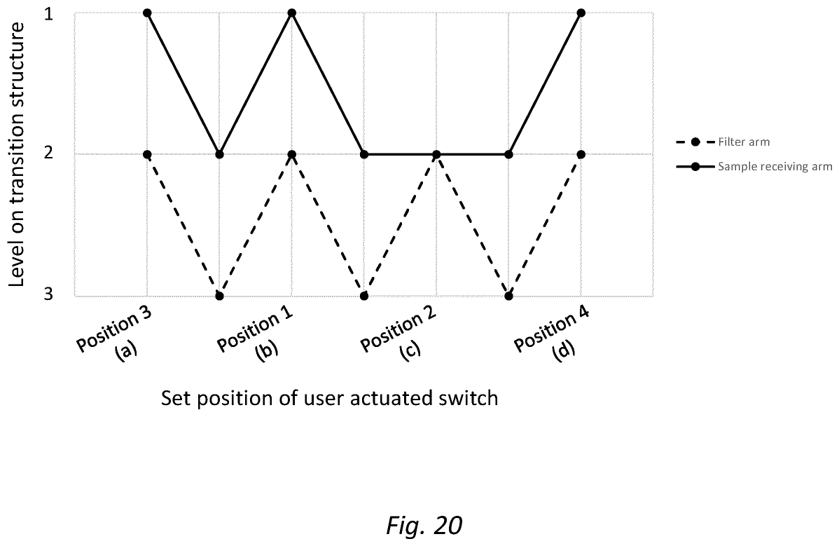

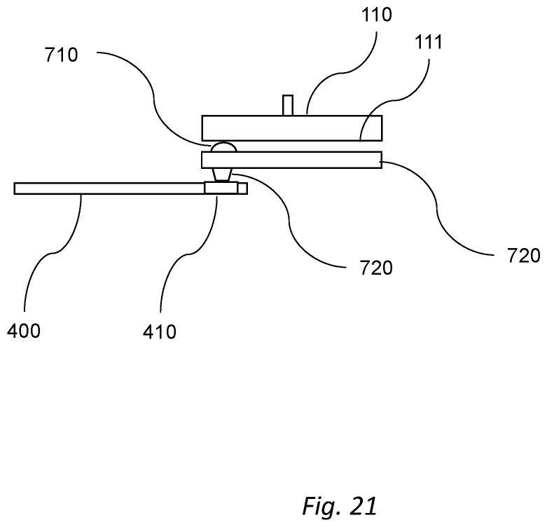

FIG. 20 shows a graph indicating the levels of the transition structure 200 at which the first contact point 430 and the second contact point 560 rest at the set positions and during the transitions between them.

At position a, the first contact point 430 (of the filter arm 400) rests on the second level 220 of the transition structure, and the second contact point 560 (of the sample receiving arm 500) rests on the first level 210. During the transition between the position a and position b, both the first contact point 430 and the second contact point 560 drop one level lower (the first contact point 430 to the third level 230 and the second contact point 560 to the second level 220) so that they avoid interference. Both the first contact point 430 and the second contact point 560 are then raised by one level (the first contact point 430 to the second level 220 and the second contact point 560 to the first level 210) so that the liquid release member 420 makes contact with the filtering buffer container 340 and creates an opening in the filtering buffer container 340 (for example by piercing it). The dial 110 continues rotating (with the first and second contact points 430 and 560 remaining at second and first levels 220 and 210 respectively) such that the liquid release member 420 moves across the filtering buffer container 340, increasing the size of the opening and releasing the filtering buffer liquid. At the end of the transition, the dial 110 reaches the position b, with the first contact point 430 resting on the second level 220 and the second contact point 560 resting on the first level 210. The liquid release member 420 rests at the edge of the filtering buffer container 340.

During the transition from position b to position c, both the first contact point 430 and the second contact point 560 drop one level lower (the first contact point 430 to the third level 230 and the second contact point 560 to the second level 220) so that they avoid interference and the liquid release member 420 is released from the filtering buffer container 340. The second contact point 560 remains at the second level 220. The first contact point 430 moves up one level to the second level 220, so that the filter aperture 410 is translated and is aligned with the residue receiving portion 520. The liquid release member 420 then makes contact with the processing buffer container 350 and creates an opening in the processing buffer container 350 (for example by piercing it). The dial 110 continues rotating (with the first and second contact points 430 and 560 remaining at second level 220) such that the liquid release member 420 moves across the processing buffer container 350, increasing the size of the opening and releasing the processing buffer liquid. At the end of the transition, the dial 110 reaches position c, with both the first contact point 430 and the second contact point 560 resting on the second level 220. The liquid release member 420 rests at the edge of the processing buffer container 350. In the above description the processing buffer liquid is released after the filter arm 400 has moved such that the filter aperture 410 is aligned with the residue receiving portion 520. In another embodiment, the processing buffer liquid may be released while the filter arm 400 is moving.

During the transition from position c to position d, the first contact point 430 drops one level lower to the third level 230, such that the liquid release member 420 exits the third buffer container 350. The second contact point 560 remains at the second level 220. Both first and second contact points 430 and 560 then move up one level (to the second and first levels 220 and 210 respectively) so that the liquid release member 420 then makes contact with the chasing buffer container 360 and creates an opening in the chasing buffer container 360 (for example by piercing it). The dial 110 continues rotating (with the first and second contact points 430 and 560 remaining at second level 220 and first level 210 respectively) such that the liquid release member 420 moves across the chasing buffer container 360, increasing the size of the opening and releasing the chasing buffer liquid. At the end of the transition, the dial 110 reaches position d, with the first contact point 430 resting on the second level 220 and the second contact point 560 resting on the first level 210. The liquid release member 420 rests at the edge of the chasing buffer container 360.

In use, at position a the user deposits a suspension into the sample receiving aperture 320. At this position, the sample receiving aperture 320 is aligned with the filter aperture 410 and the filtrate receiving portion 510. The user moves the dial 110 position a to position b, and in doing so the filtering buffer liquid is released. The filtering buffer liquid washes the filtrate through the filter membrane in the filter aperture 410, leaving the washed residue on the filter membrane. The filtering buffer liquid chases the filtrate into the filtrate receiving portion 510. The user then turns the dial 110 from position b to position c. This causes the filter aperture 410 to move to be aligned with the residue receiving portion 520, after which the processing buffer liquid is released from the processing buffer container. The processing buffer liquid breaks down the residue on the filter membrane, releasing a residue component that can pass through the filter membrane. Finally, the user turns the dial from position c to position d, releasing the chasing buffer liquid that chases the residue component into the residue receiving portion.

In an embodiment, the filtrate receiving portion 510 and the residue receiving portion 520 comprise lateral flow strips. The filtering buffer liquid chases the filtrate along the filtrate a first lateral flow strip, and the chasing buffer liquid chases the residue component along a second lateral flow strip. In an example, the suspension may be blood. The first lateral flow strip may test for a sepsis marker (for example lactate or C-reactive protein CRP) in the filtrate (plasma). The processing buffer liquid lyses the white blood cells, releasing proteins (cellular CD16b). The chasing buffer liquid then chases these proteins down the second lateral flow strip, which measures the CD16b (indicative of white blood cell count). The two lateral flow strips may indicate the sepsis marker level or neutrophil level by matching the intensity of the line formed on the lateral flow strip to a key. The lateral flow strips may also show a control line. The two assays will be discussed in more detail later in the description.

The results of the lateral flow tests may be viewed through the apertures 130 on the top 610 of the housing 120, or in another way.

In an alternative embodiment, where the residue does not require both further processing and a chasing buffer, the fourth set position and the chasing buffer liquid may not be required. In this embodiment the assay device may not comprise the chasing buffer container 360. The user may turn the dial 110 from position a to position b, releasing the filtering buffer liquid to chase the filtrate into the filtrate receiving portion 510. The user may then turn the dial position b to position c, moving the filter aperture to the residue receiving portion 520. The processing buffer liquid may be released processing the residue to allow a residue component to pass through the filter membrane and chasing the processed residue component into the residue receiving portion 520.

In an alternative embodiment, the filtrate may not require a filtering buffer liquid (for example, if the viscosity and volume of the filtrate may be such that it travels into the filtrate receiving portion without assistance from a buffer). In this embodiment the assay device may not comprise the filtering buffer container 340. The user may deposit the sample with the dial at position b. The filtrate may pass into the filtrate receiving portion 510. The user may then turn the dial 110 from position b to position c, moving the filter aperture to the residue receiving portion 520. The processing buffer liquid may be released, processing the residue to allow a residue component to pass through the filter membrane. The user may then turn the dial 110 from position c to position d, releasing the chasing buffer. The chasing buffer may wash the processed residue component into the residue receiving portion 520.

Tests may be performed on the filtrate, or the residue, or both. The filtrate receiving portion 510 and the residue receiving portion 520 may each comprise a lateral flow test strip or a lateral flow dummy strip. The filtrate receiving portion 510 may direct the filtrate to microfluidics. The residue receiving portion 520 may direct the residue to microfluidics. The residue may be eluted from the filter membrane, for example into a vessel or onto a device or test strip. Other assays may be used.

The timings of the transitions are controlled by the geometry of the plurality of levels and the ramps of the transition structure 200. This is a factor in achieving precise timings and volumes of released buffer solutions. In the example given above, the lysing buffer may need a certain amount of time in contact with the cells before the chasing buffer is added.

The user actuated switch may be configured to operate in one direction only, such that the user can move the switch between the set positions only in the intended order. The assay device may comprise a single use snap. The plurality of levels may comprise notches to prevent backwards travel.

The user actuated switch may comprise a dial (wherein the plurality of levels comprise concentric arcs), a slider (wherein the plurality of levels comprise parallel strips), or other mechanism.

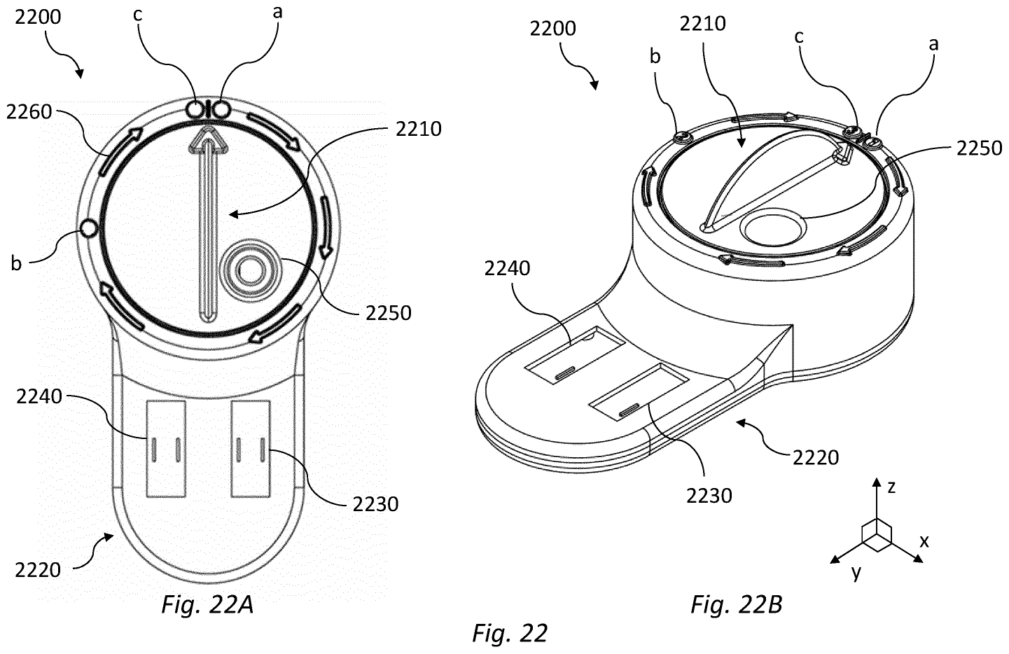

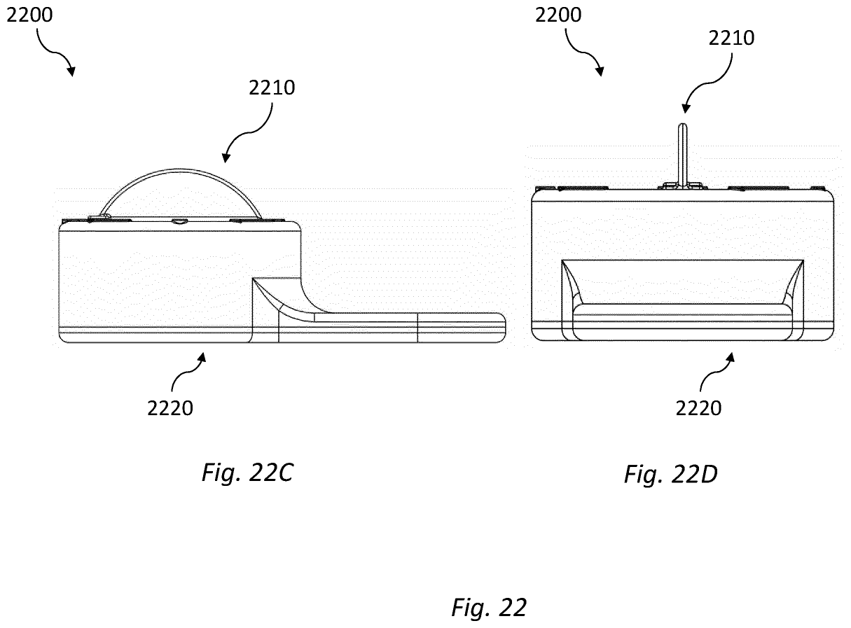

The liquid release member 420 in the example discussed above comprises a spike to pierce the buffer containers. The buffer containers may comprise apertures in the dial 110 containing capsules that are configured to be pierced by the liquid release member. The liquid release member may create an opening in the buffer container by other means, for example by exerting pressure in order to burst the buffer container. With reference to FIG. 22, the buffer containers may comprise buffer pods 710 (in holder 720) that are positioned between the user actuated switch 110 and the filter arm 400. The user actuated switch may comprise one or more actuating features on the lower side 111 of the user actuated switch 110, which are configured to make contact with the buffer pod 710 when the user actuated switch is moved and burst the buffer pod 710. The buffer liquid may then be directed to the filter aperture 410, for example via a nozzle 730. The buffer pod 710 may be configured to burst via a localised opening points. In this embodiment, the sample receiving arm 500 may be fixed and not guided by the transition structure 200.

### Second Specific Embodiment

A second specific embodiment of the disclosure will now be described, by way of example. It will be understood from the preceding description that this disclosure covers both this second specific example, and other examples.

FIG. 22 shows a top view (FIG. 22A) and a perspective view (FIG. 22B) of a second specific embodiment of an assay device 2200 in accordance with the disclosure. The assay device 2200 comprises a user actuated switch comprising a dial 2210. The assay device 2200 comprises a housing 2220. In the example shown in FIG. 22, the housing comprises apertures 2230 and 2240 for viewing results of testing the suspension. The assay device 2200 may, for example, comprise a first and second lateral flow strip that would be visible through apertures 2230 and 2240. The assay device 2200 of the second specific embodiment comprises first and second buffer containers (not visible in FIG. 22). The user actuated switch is movable between first, second and third set positions. In FIG. 22, the third set position is marked as “a”, the first set position is marked as “b” and the second set position is marked as “c”. The set positions may be marked in any appropriate way. The dial further comprises a sample receiving aperture 2250, via which a user may deposit a sample. The direction of rotation of the dial 2210 is shown by the arrows around the edge of the dial (for example arrow 2260 between b and c). These arrows may or may not be marked on the device. FIG. 22B also shows axes indicating the vertical direction z and the horizontal plane x-y to assist subsequent descriptions. FIG. 22C shows a side view of the assay device 2200 in the z-y plane, and FIG. 22D shows a side view of the assay device in the z-x plane.

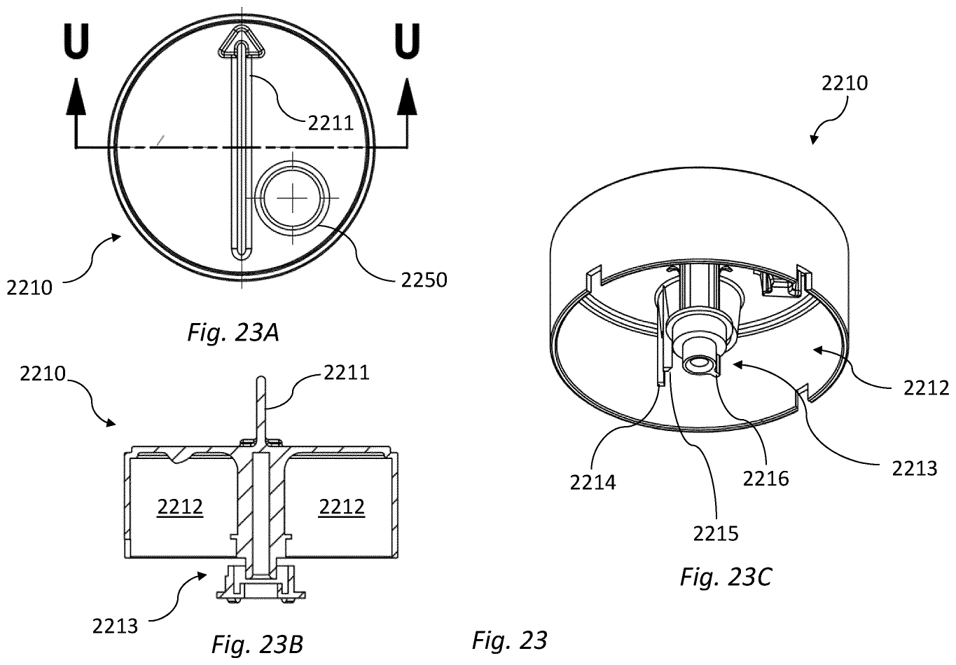

With reference to FIGS. 23, several views of the dial 2210 in isolation are shown. FIG. 23A shows a top view of the dial 2210. The dial 2210 of this example comprises a raised portion 2211 configured to be gripped by the user actuating the user actuated switch. In other embodiments, the dial may not comprise a raised portion. For example, the dial may be turned by gripping the edges of the dial. The dial 2210 further comprises the sample receiving aperture 2250, via which the user may deposit the sample. The dial 2210 has a circular cross-section in the x-y plane. FIG. 23B shows a cross-section of the dial shown in FIG. 23A, taken along the line U-U and in the z-x plane. The dial 2210 comprises a void 2212, within which other components can rotate as described later, and a connection 2213 configured to rotatably connect the dial 2210 to other components described later. FIG. 23C shows a perspective view of the dial below, showing the void 2212 and the connection 2213. FIG. 23C also shows sacrificial ribs 2314 and 2315 and protrusion 2316 on connection 2213, which will be discussed later

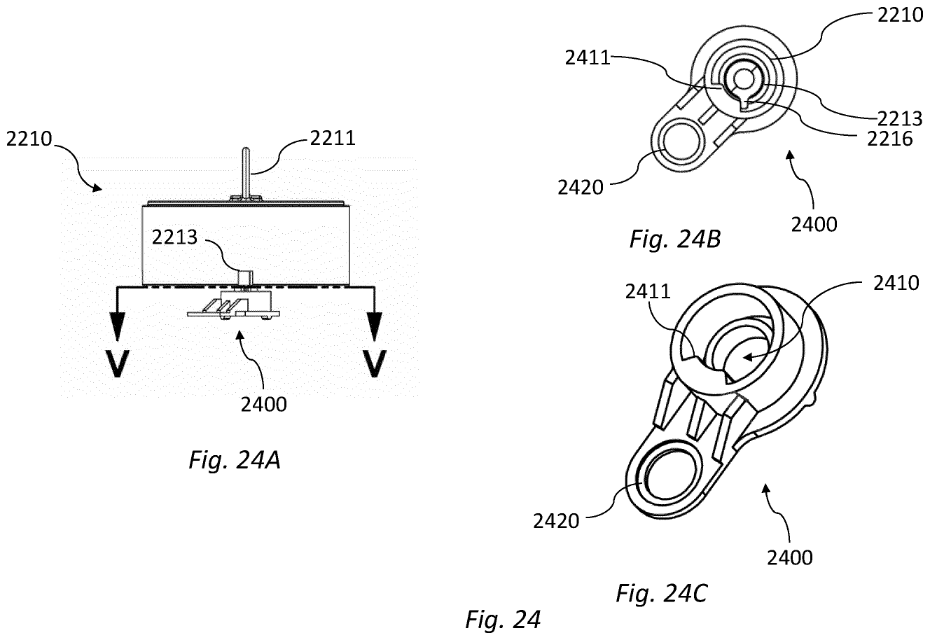

With reference to FIG. 24, the assay device 2200 further comprises a filter arm 2400, wherein the filter arm 2400 is configured to be guided by the transition structure of the dial 2210. The filter arm 2400 is configured to be rotatably coupled to the dial 2210 via the connection 2213 of the dial. FIG. 24A shows a side view of the dial 2210. The filter arm 2400 is shown to be underneath the dial 2210. FIG. 24B shows a top view of the filter arm 2400 from an x-y plane along the line V-V of FIG. 24A. FIG. 24C shows a perspective view of the filter arm 2400. The filter arm 2400 comprises a connection aperture 2410 configured to couple rotatably to the connection 2213 of the dial, such that the filter arm is rotatable about the same axis as the dial 2210. The connection 2213 may pass through the connection aperture 2410. The cross-section of FIG. 24B shows the connection 2213 in the connection aperture 2410. In the position shown in FIG. 24B, the protrusion 2216 on the connection 2213 rests against a protrusion 2411 in the connection aperture 2410, so that further rotation of the dial in a clockwise direction will rotate the filter arm. This occurs in the transition between position b and position c, as described later. In other positions, the protrusion 2216 of the connection 2213 of the dial does not rest against the protrusion in the connection aperture 2410, so rotating the dial does not rotate the filter arm 2400. The filter arm further comprises a filter aperture 2420 configured to accommodate a filter membrane. In use, the filter aperture may contain a filter membrane.

The assay device 2200 will now be further described in the context of the set positions and transitions between the set positions.

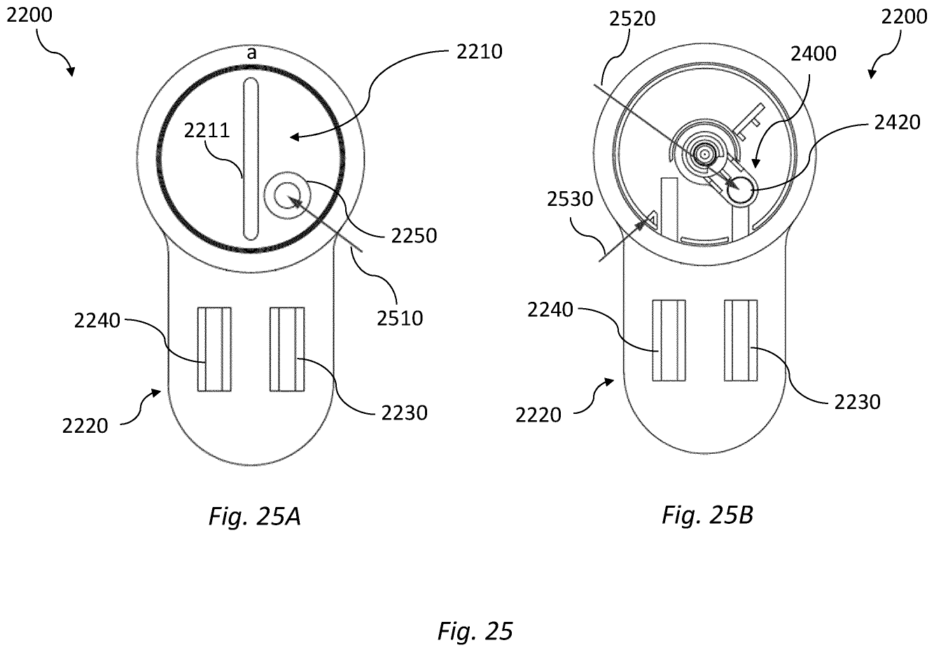

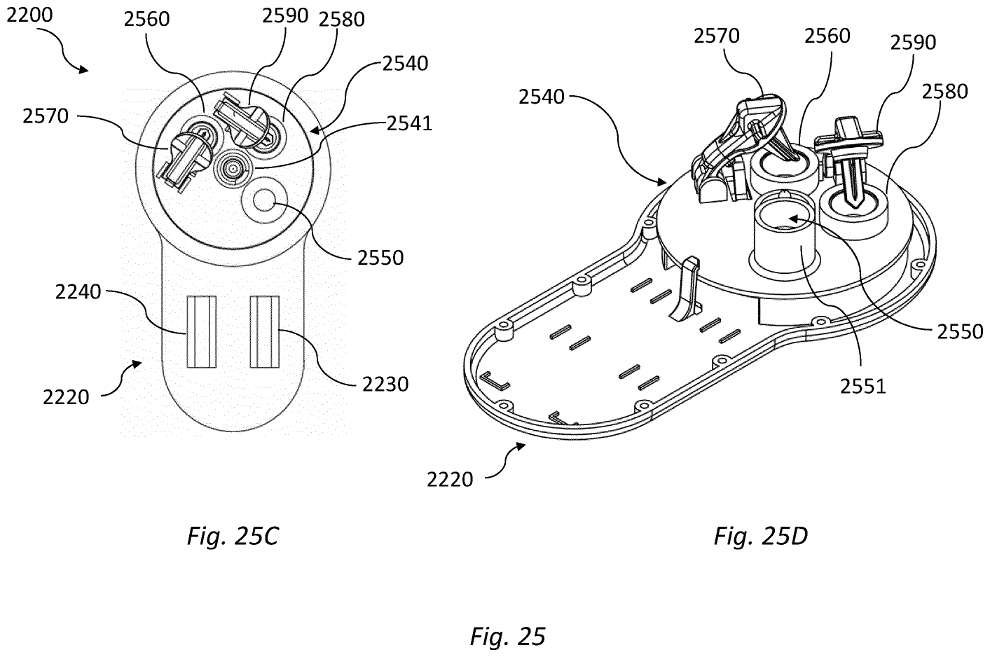

With reference to FIG. 25, the assay device 2200 is shown in the third set position (also referred to as position a). FIG. 25A shows a top view of the assay device 2200. In use, the assay device 2200 starts in position a. The user deposits a sample, such a blood sample, into sample receiving aperture 2250, as indicated by arrow 2510. FIG. 25B shows a top view of the assay device 2200, with upper components removed to leave the holder 2220 and the filter arm 2400. At position a, the sample receiving aperture 2250 aligns with the filter aperture 2420. The filter aperture 2420 aligns with the filtrate receiving portion. The filtrate receiving portion may comprise a test strip, wherein results on the test strip may be viewed via aperture 2230. In use, when the user deposits a sample (comprising a filtrate and a residue) into sample receiving aperture 2250, the sample is received by the filter aperture 2420 (as indicated by arrow 2520). Arrow 2530 indicates an end stop, wherein the starting position a may be found by the user by rotating the dial anti-clockwise until the dial makes contact with the end stop.

FIGS. 25C and 25D show the assay device 2200 at the same position a, but show the housing 2220 and a buffer cassette 2540 configured to be rotatable about the same axis as the dial 2210 and the filter arm 2400. FIG. 25C shows a top view of the assay device 2200, with the buffer cassette 2540 and a top portion of the housing 2220 comprising the apertures 2230 and 2240. FIG. 25D shows a perspective view of the assay device 2200 with the buffer cassette 2540 but with only a lower portion of the housing 2220 (without the top portion). The filter arm 2400, not shown, would be arranged below the buffer cassette 2540. The connection 2213 of the dial 2210 is configured to rotatably couple to the buffer cassette 2540 via a connection aperture 2541. The connection 2213 of the dial 2210 may pass through the connection aperture 2541 of the buffer cassette 2540, pass through connection aperture 2410 of the filter arm 2400, and connect rotatably to the housing 2220.

The buffer cassette 2540 comprises an aperture 2550. At position a, the aperture 2550 is aligned with the sample receiving aperture 2250, the filter aperture 2420, and the filtrate receiving portion. In use, when a user deposits the sample in the sample receiving aperture 2250 of the dial, the sample passes through the aperture 2550 to the filter aperture 2420. The aperture 2550 may comprise a wall 2551 configured to guide the sample to the filter aperture 2420. The buffer cassette 2540 further comprises a filtering buffer container 2560 (also referred to as the second buffer container), a primary piercer 2570 (also referred to as the second liquid release member) configured to create an outlet of the filtering buffer container 2560, a processing buffer container 2580 (also referred to as the first buffer container) and a secondary piercer 2590 (also referred to as the first liquid release member) configured to create an outlet of the processing buffer container 2580. The primary and secondary piercers 2570 and 2590 may each comprise a spike configured to create an outlet of a buffer container, and may be hingedly connected to the buffer cassette such that the primary and secondary piercers 2570 and 2590 may be configured to be in a raised position or a lowered position, wherein pushing the primary and secondary piercers 2570 and 2590 from the raised position to the lowered position creates an outlet in the filtering and processing buffer containers 2560 and 2580 respectively.

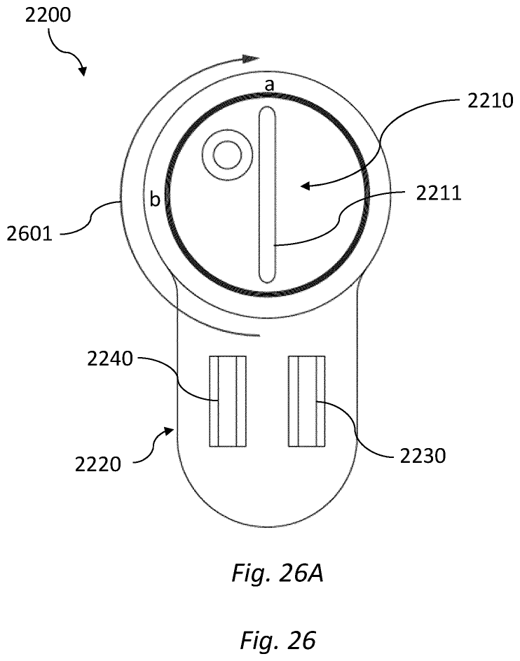

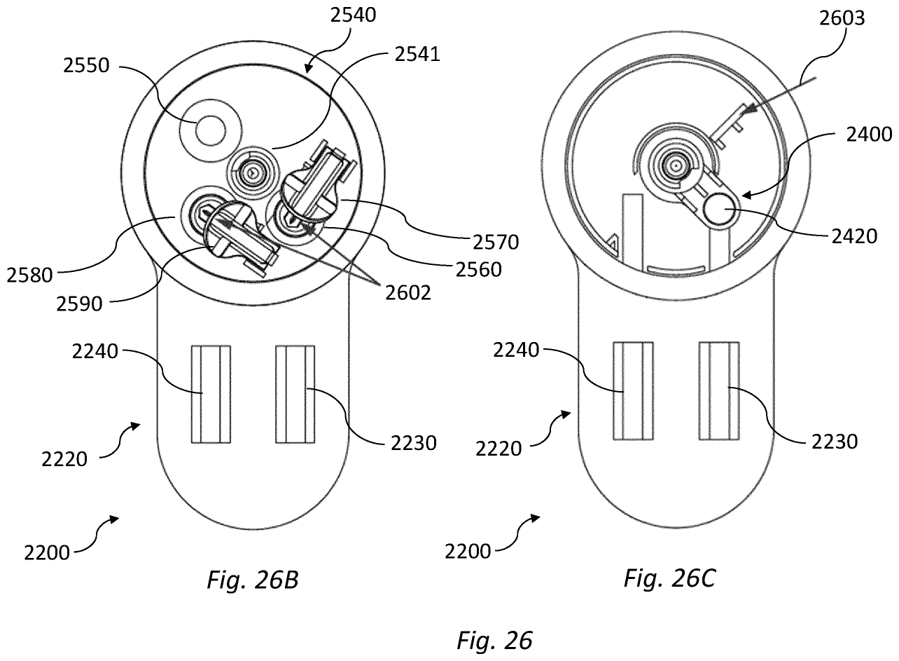

In use, the user actuates the dial to move the dial from the third set position (position a) to the first set position (also referred to as position b). The transition from position a to position b may comprise two stages, although the user may turn the dial from a to b in one continuous motion. The user will turn the dial by 270° during this transition, but we will discuss this as being split into a first stage of 180° and a second stage of 90° (experienced by the user as a continuous 270° turn). With reference to FIG. 26, the end of the 180° stage is shown. FIG. 26A shows a top view of the assay device, with the curved arrow 2601 indicating the 180° turn that the dial has made. FIG. 26B shows a top view of assay device 2200 in the same position as FIG. 26A, with the dial 2210 removed to show the buffer cassette 2540. The filtering buffer container 2560 is aligned with the filtrate receiving portion and the processing buffer container 2580 is aligned with the residue receiving portion. Both the first and secondary piercers 2570 and 2590 are raised (indicated by arrows 2602). FIG. 26C shows the assay device 2200 in the position shown in FIGS. 26A and B, with the buffer cassette 2540 removed to show the filter arm 2400. The filter aperture 2420 is aligned with the filtrate receiving portion and with the filtering buffer container 2560. Sacrificial ribs 2214 and 2215 on the underside of the dial 2210 may rotate the buffer cassette 2540 during the 180° of the dial to align the buffer cassette in this way. The buffer cassette will remain at this alignment for the remainder of the use of the assay device 2200, so a buffer end stop (indicated by arrow 2603) is configured to prevent the buffer cassette from rotating further when the dial is turned further than the position shown in FIG. 26A.

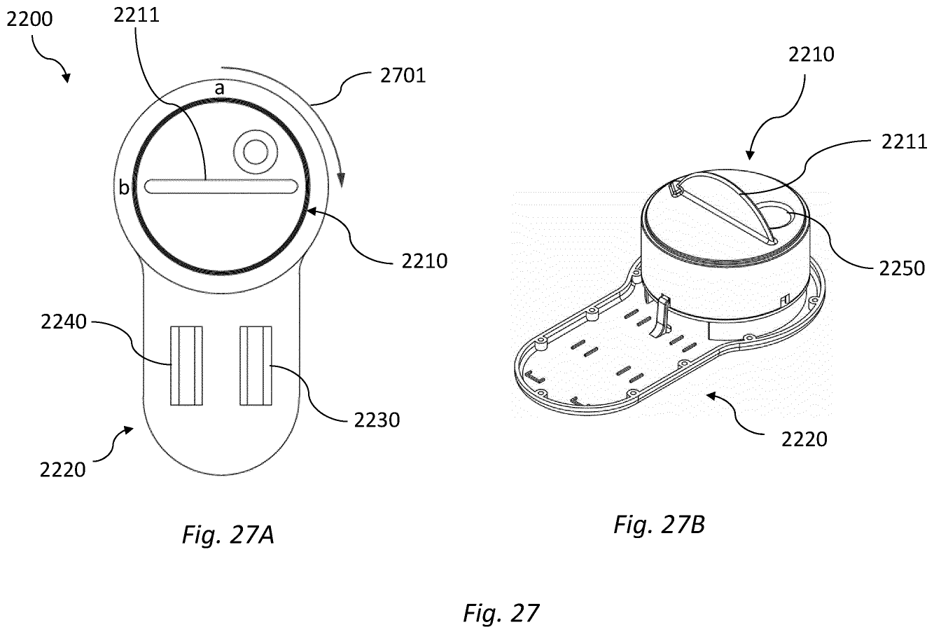

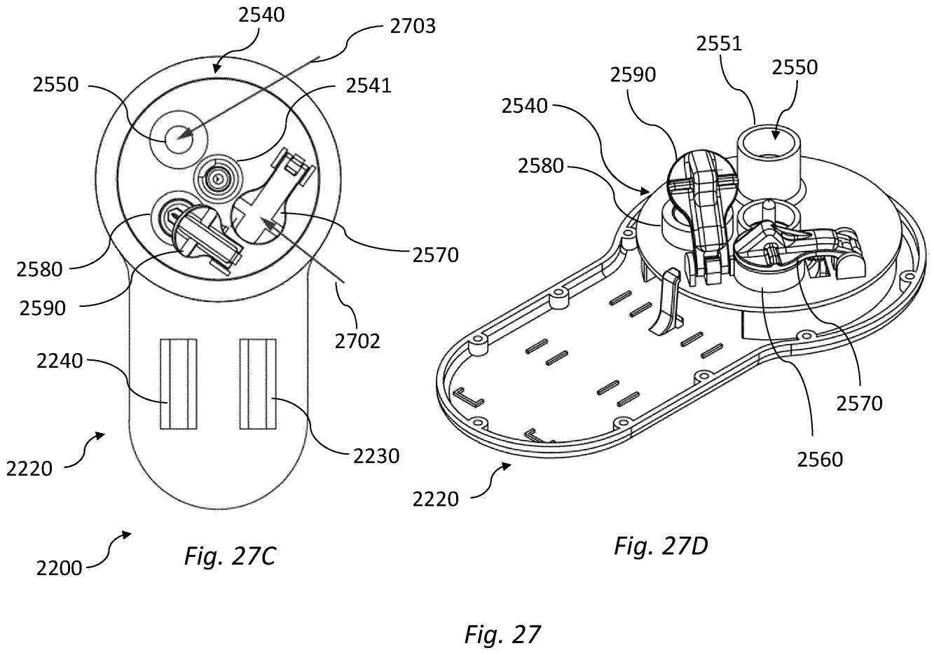

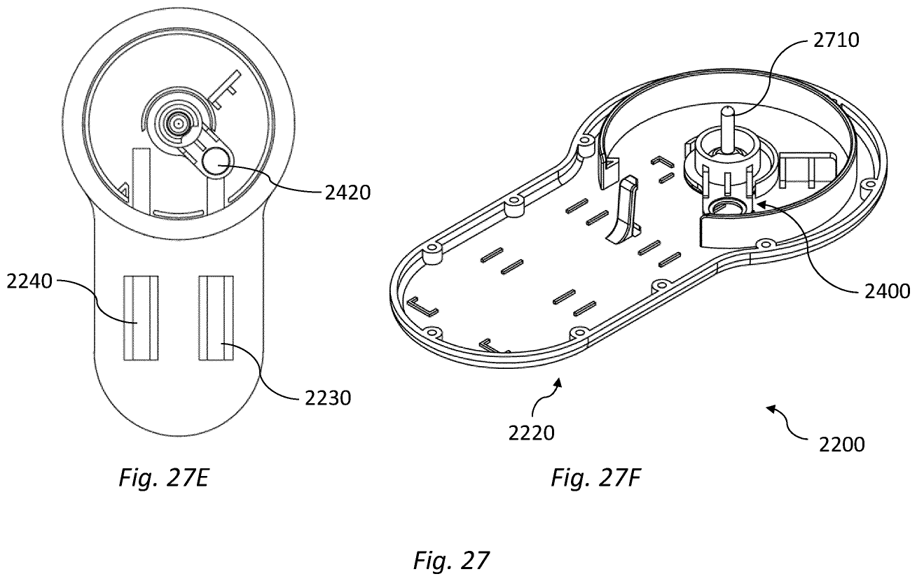

FIG. 27 shows the assay device 2200 at position b, after the full 270° rotation of the dial from position a. FIG. 27A shows a top view of the assay device, with the curved arrow 2701 indicating the 90° turn that the dial has made from the position shown in FIG. 26. FIG. 27B shows a perspective view of the assay device 2200 with the dial 2210 in the position shown in FIG. 27A, without the top portion of the housing 2220. FIG. 27C shows a top view of the assay device at position b with the dial 2210 removed to show the buffer cassette 2540. FIG. 27D shows a perspective view of the assay device at position b with the dial 2210 removed to show the buffer cassette 2540, and the upper portion of the housing removed. FIG. 27E shows a top view of the assay device at position b with the dial 2210 and the buffer cassette 2540 removed to show the filter arm 2400, wherein the filter arm 2400 is aligned with the filtrate receiving portion. FIG. 27F shows a perspective view of the assay device at position b with the dial 2210 and the buffer cassette 2540 removed to show the filter arm 2400.

At position b, the filtering buffer container 2560 is aligned with the filtrate receiving portion and the processing buffer container 2580 is aligned with the residue receiving portion, as in the stage shown in FIG. 26. During the transition from the position shown in FIG. 26 to position b, the wall 2551 of the aperture 2550 of the buffer cassette 2540 may break the sacrificial ribs 2214 and 2215 on the underside of the dial that were used to rotate the buffer cassette 2540. Breaking the sacrificial ribs 2214 and 2215 allows the piercers to actuate. The primary piercer may be actuated by an actuating post on the underside of the dial, wherein breaking the sacrificial ribs 2214 and 2215 allows the actuating post to interact with the primary piercer 2570. At position b the primary piercer 2570 is lowered such that it creates an outlet in the filtering buffer container 2560. In an example, the filtering buffer container 2560 may comprise an upper and lower film so the primary piercer 2570 may pierce both the upper and lower films so that the filtering fluid in the filtering buffer container 2560 flows down to the filter aperture 2420. The primary piercer may make contact with a filter membrane in the filter aperture, such that the filtering fluid is transferred to (and through) the filter membrane via wicking. The films may, for example, comprise foil. In use, the liquid contained within the filtering buffer container 2560 flows through the filter aperture 2420 and onto the filtrate receiving portion, washing the filtrate of the sample to the filtrate receiving portion. In an example, the filtrate receiving portion comprises a lateral flow strip, and the liquid contained within the filtering buffer container (such as a chase buffer) washes the filtrate along the lateral flow strip. FIG. 27F shows a pin 2710, which may be configured to rotatably couple the connection 2213 of the dial to the housing 2220.

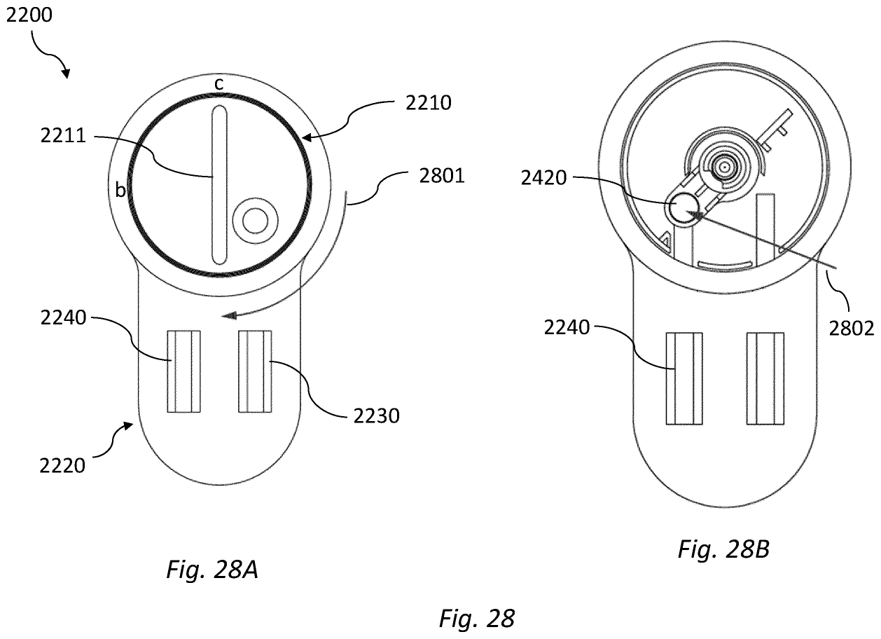

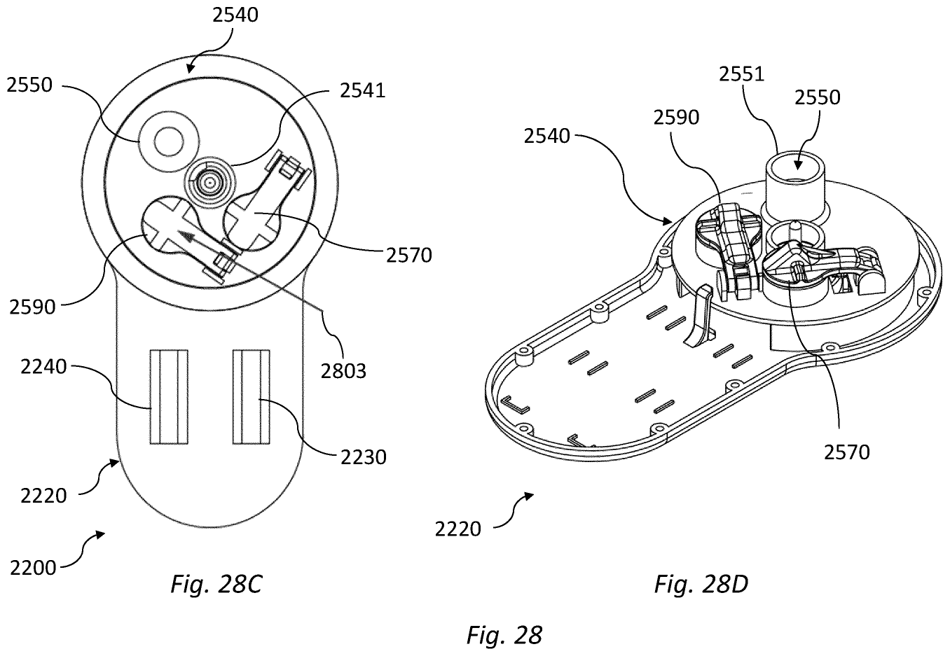

With reference to FIG. 28, a top view of the assay device 2200 in position c (second set position) is shown. In use, the dial is rotated by 90° during the transition from position b to position c (indicated by arrow 2801). FIG. 28B shows a top view of the assay device at position c with the dial 2210 and the buffer cassette 2540 removed to show the filter arm 2400. FIG. 28C shows a top view of the assay device at position c with the dial 2210 removed to show the buffer cassette 2540. FIG. 28D shows a perspective view of the assay device at position c with the dial 2210 removed to show the buffer cassette 2540, and the upper portion of the housing removed.

During the transition from position b to position c, the filter arm is rotated such that at position c the filter aperture is aligned with the residue receiving portion (as indicated by arrow 2802 in FIG. 28B) and with the processing buffer capsule 2580 (shown in FIG. 28C). Referring back to FIGS. 23C and 24B, at position b a protrusion 2216 on the connection 2213 of the dial 2210 rests against a protrusion 2411 of the connection aperture 2410 of the filter arm 2400, so rotating the dial 2210 clockwise rotates the filter arm 2410 clockwise. The secondary piercer 2590 may be lowered (indicated by arrow 2803) during the transition from position b to position c such that it creates an outlet in the processing buffer container 2580. In an example, the processing buffer container 2580 may comprise an upper and lower film so the secondary piercer may pierce both the upper and lower films so that the processing fluid in the processing buffer container 2580 flows down to the filter aperture 2420. The secondary piercer may make contact with a filter membrane in the filter aperture, such that the processing fluid is transferred to (and through) the filter membrane via wicking. The films may, for example, comprise foil. In use, the liquid contained within the processing buffer container 2580 flows through the filter aperture 2420 and processes the residue (for example via lysis) such that the residue can pass through a filter membrane in the filter aperture 2420 and onto the residue receiving portion. In an example, the residue receiving portion comprises a lateral flow strip, and the liquid contained within the processing buffer container (such as a lysis buffer) washes the processed residue along the lateral flow strip. The primary piercer 2570 may remain lowered as at position b.

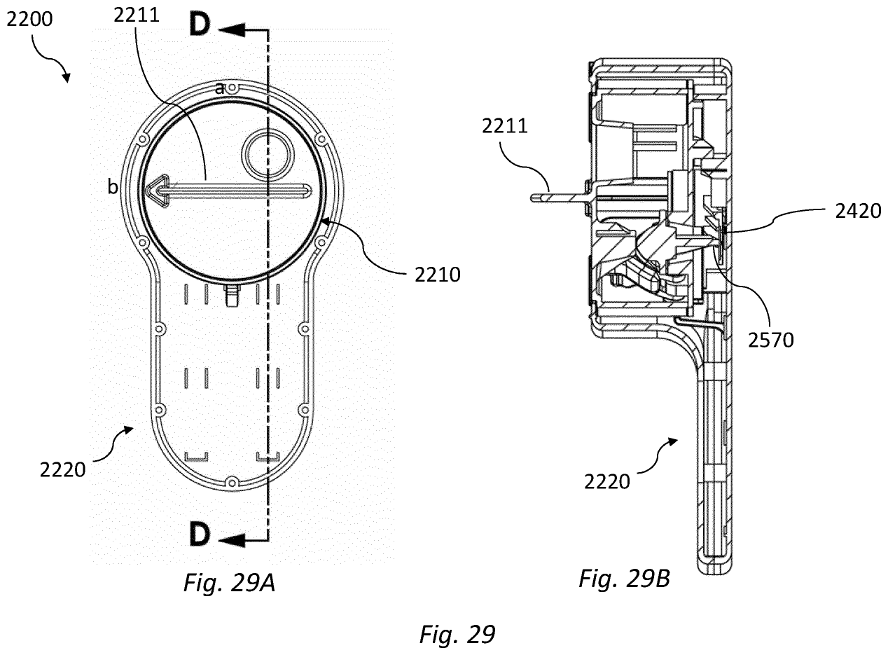

FIGS. 29 to 34 show detailed views of some components of the assay device 2200.

FIG. 29 shows the device at position b, wherein the filter aperture 2420 and filtering buffer container 2560 are aligned with the filtrate receiving portion, the processing buffer container 2580 is aligned with the residue receiving portion, and the primary piercer 2570 is lowered such that an outlet in the filtering buffer container 2560 is created. FIG. 29A shows a top view of the assay device 2200 with the upper portion of the housing removed. FIG. 29B shows a cross-section taken along the line D-D in FIG. 29A, in the z-y plane. A tip of the primary piercer 2570 makes contact with (but does not pierce) a filter membrane located in the filter aperture 2420. The liquid within the filtering buffer container 2560 may travel down the primary piercer 2570, for example via wicking, to reach the filter aperture 2420. The primary piercer 2570 may be shaped to assist liquid movement. For example, the primary piercer 2570 may be smooth, ridged, serrated, or some other shape.

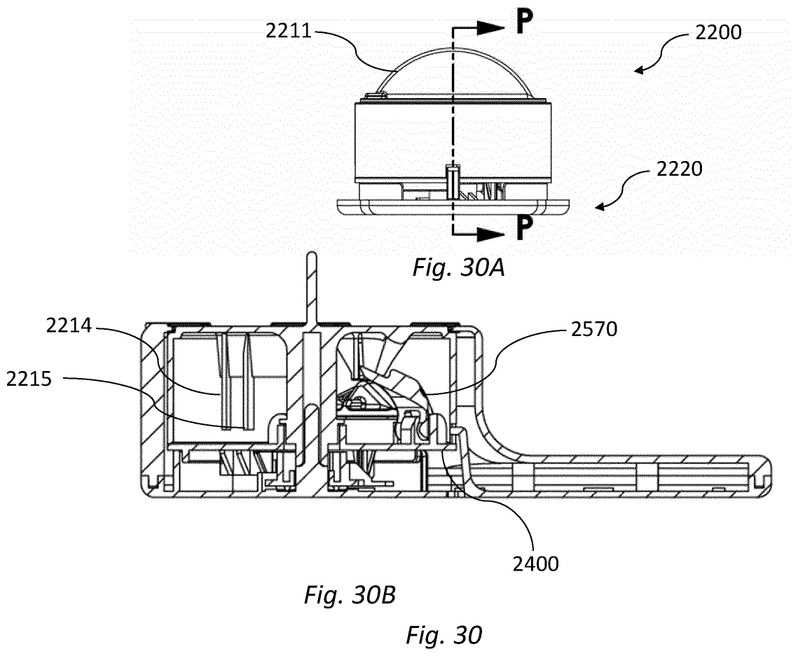

FIG. 30 shows the device during the transition from position a to b, wherein the filter aperture 2420 and filtering buffer container 2560 are aligned with the filtrate receiving portion, the processing buffer container 2580 is aligned with the residue receiving portion, but the primary piercer 2570 is not yet lowered such that an outlet in the filtering buffer container 2560 is created. The sacrificial ribs 2214 and 2215 on the underside of the dial 2210 that are configured to rotate the buffer cassette 2540 have not yet been broken to allow the actuation of the primary piercer. FIG. 30A shows a side view of the assay device 2200 in the z-x plane. FIG. 30B shows a cross-section of the assay device 2200 taken along the line P-P of FIG. 30A, in the z-y plane. The sacrificial ribs 2214 and 2215 are shown (there may be more or fewer sacrificial ribs). Filter arm 2400 is also labelled, aligned with the filtrate receiving portion and with a raised primary piercer 2570.

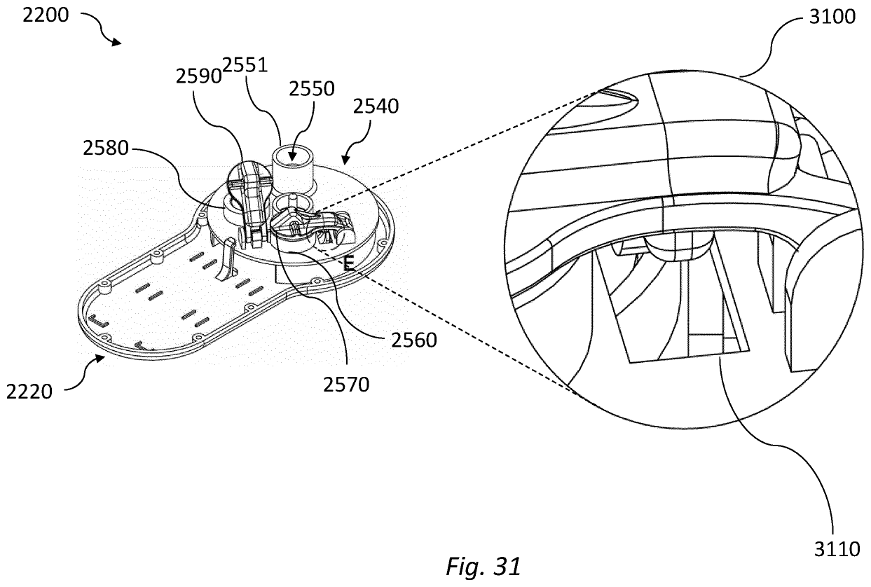

FIG. 31 shows the assay device 2200 in during the transition from position b to position c. FIG. 31 shows a perspective view of the assay device 2200 with dial and upper portion of the housing removed to show the buffer cassette 2540. The filtering buffer container 2560 is aligned with the filtrate receiving portion, the processing buffer container 2580 is aligned with the residue receiving portion, and the primary piercer 2570 is lowered. During the transition from position b to position c, the filter arm 2400 rotates from being aligned with the filtrate receiving portion to being aligned with the residue receiving portion. At position b the primary piercer 2570 is in contact with a filter membrane in the filter aperture, so during the transition from position b to position c the primary piercer 2570 is raised slightly. The pressure on the actuating post that lowered the primary piercer 2570 is relieved slightly, and a small, moulded spring pushes the primary piercer 2570 up slightly such that it is not in contact with the filter membrane. The magnified portion 3100 of FIG. 31 shows the primary piercer being pushed up through an aperture 3110 in the buffer cassette 2540.

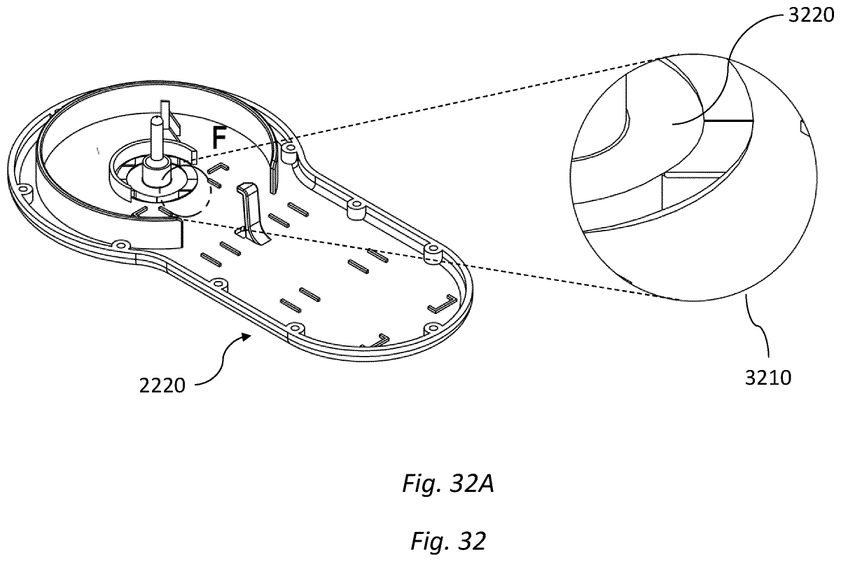

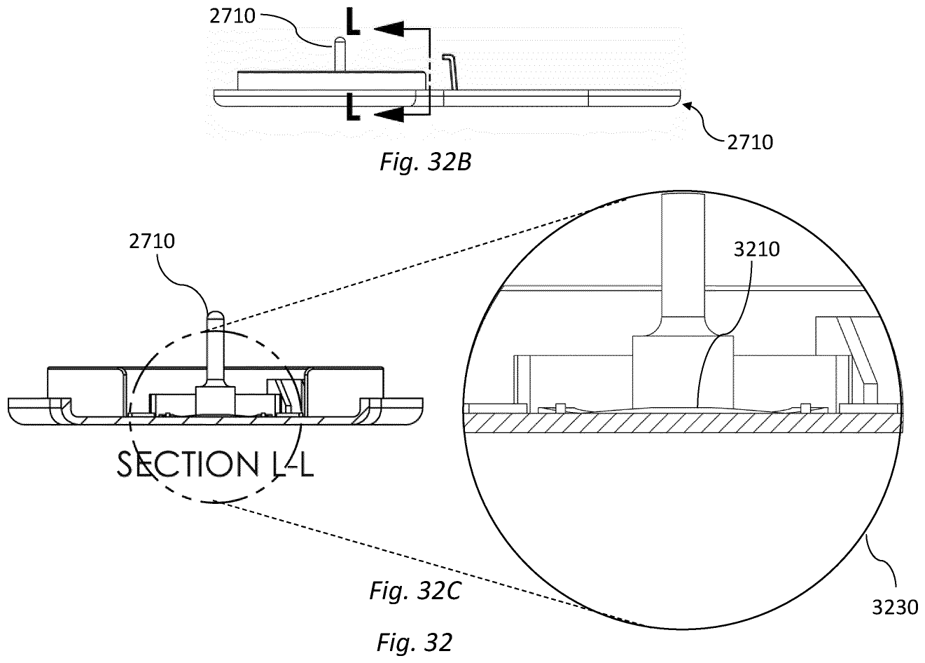

With reference to FIG. 32, during the transition from position b to position c, the filter arm 2400 is raised slightly. During the transition a ramp on the lower portion of the housing raises and lowers the filter arm 2400, such that at position c the filter arm 2400 is at the same level as at position b. FIG. 32A shows a perspective view of the lower portion of the housing 2220. The magnified portion 3210 of FIG. 32A shows the ramp 3220. FIG. 32B shows a side view of the lower portion of the housing 2220 shown in FIG. 32A. FIG. 32C shows a cross-sectional view taken along line L-L of FIG. 32B, in the z-x plane and in the direction of the arrows of the L-L line (i.e. looking towards the pin 2710). The magnified portion 3230 of FIG. 32C shows the ramp 3210. The filter arm 2400 may comprise feet configured to rest on the ramp 3220, such that during the transition from position b to position c the feet move along the ramp, raising and lowering the filter arm 2400. In other examples, the filter arm may be configured to rest on another shape of ridge or track such that during the transition from position b to position c the filter arm 2400 moves along the ramp, raising and lowering the filter arm 2400. The transition structure may comprise a plurality of levels. The filter arm 2400 may be at a plurality of levels at different set positions and/or during transitions between set positions.

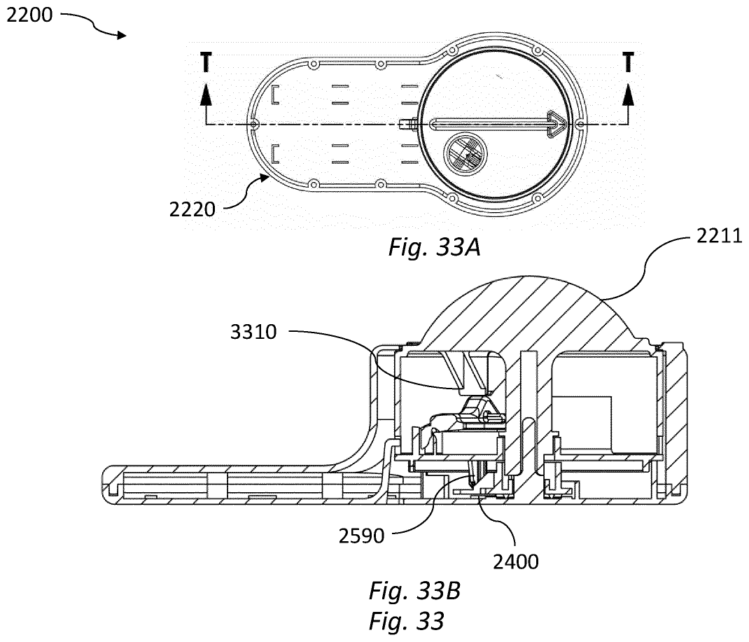

As described earlier, the first and secondary piercers may be pushed down by an actuating post on the underside of the dial 2210. With reference to FIG. 33, FIG. 33A shows a top view of the assay device 2200 with the upper portion of the housing 2220 removed. The assay device is in position c. FIG. 33B shows a cross-section of the assay device 2200 shown in FIG. 33A, taken along the line T-T in the z-y plane. Actuating post 3310 is shown pushing down the secondary piercer 2590 such that an end of the secondary piercer 2590 is in contact with a filter membrane located in the filter aperture 2420 of the filter arm 2400.

In an embodiment, the filtrate receiving portion and the residue receiving portion comprise lateral flow strips. In an example, the suspension may be blood. The first lateral flow strip may test for a sepsis marker such as lactate or C-reactive protein (CRP) in the filtrate (plasma). The filtering buffer liquid chases the filtrate along the filtrate lateral flow strip. The processing buffer liquid lyses the white blood cells, releasing proteins (cellular CD16b). The processing buffer liquid then chases these proteins down the second lateral flow strip, which measures the CD16b (indicative of white blood cell count). The two lateral flow strips may indicate the sepsis marker level or neutrophil level by matching the intensity of the line formed on the lateral flow strip to a key. The lateral flow strips may also show a control line.

The two assays will be discussed in more detail in the following paragraphs. The description of the assays may apply to both the first and second specific examples of the assay device, and to other examples.

### Example of a Lateral Flow Test

An example of a lateral flow test carried out by an assay device according to embodiments of the present disclosure will now be described.

A lateral flow test may comprise a conjugate pad which may contain detection moieties such as labelled binding reagents; a suitable membrane, such as nitrocellulose, comprising a test capture line and optionally a control capture line; and optionally an absorbent pad. In use, the sample containing unknown concentrations of analyte may travel to the conjugate pad where the analyte binds to the detection moiety and travels up the nitrocellulose membrane towards the capture lines. The test line comprises an immobilised binding reagent specific for the target analyte. If present in the sample, the analyte (bound to the detection moiety) will form a complex with immobilised binding reagent resulting in a visible test line. The control line, if present, comprises an immobilised binding reagent specific for a labelled control reagent. A visible label at the control line confirms that the test has run successfully. The presence and amount of label at the capture line(s) may be determined by eye or using a suitable lateral flow device reader such as the Cube (Optricon, Germany).

The first assay analyses the soluble component in the plasma via an enzyme linked assay. Measuring sepsis marker levels (such as lactate levels or CRP levels) in the plasma may be used to test for sepsis. The filtrate receiving portion 510 may comprise a sample pad to which the sample may be added; a conjugate pad which may contain detection moieties such as labelled binding reagents; a suitable membrane, such as nitrocellulose, comprising a test capture line and optionally a control capture line; and optionally an absorbent pad.

The second assay measures the cellular component of the sample. The white blood cells may be lysed and run through an antibody based assay. The residue receiving portion 520 may not comprise a sample pad. The residue receiving portion 520 may comprise a conjugate pad which may contain detection moieties such as labelled binding reagents; a suitable membrane, such as nitrocellulose, comprising a test capture line and optionally a control capture line; and optionally an absorbent pad. The test is for the protein CD16b, which correlates to the neutrophil levels in the blood.

CD16 (a cluster of differentiation molecule found on the surface of certain white blood cells) is an IgG cell surface receptor. It is a type III Fcγ receptor (fragment, crystallisable). In humans it exists in two relatively homologous forms—CD16a and CD16b (also known as FcγRIIIa and FcγRIIIb respectively). CD16a is a transmembrane protein, whereas CD16b is anchored by a glycosyl-phosphatidylinositol (GPI) linker to the plasma membrane (Zhang et al., 2000).

CD16 proteins are expressed on the surface of neutrophils in over 99% of the population. CD16 functions in phagocytosis, degranulation, and oxidative burst. CD16b also specifically functions in removal of soluble immune complexes from blood vasculature. CD16b is expressed on neutrophils and to a far lesser extent on basophils and activated eosinophils, which comprise only 1-5% of all white blood cells.

There is a good positive correlation between the levels of CD16b and neutrophil counts, and so CD16b may be used as a neutrophil cell marker. CD16b may comprise, consist essentially of, or consist of the membrane-anchored form of CD16b (more particularly the glycosylphosphatidylinositol (GPI) anchored form of CD16b) and/or the intracellular form of CD16b. To facilitate the detection of the intracellular form of CD16b, the cell lysis may release the intracellular form of CD16b, or otherwise make it accessible to the detection moiety, such as an anti-CD16b antibody. Cell lysis may, for example, be achieved through the use of a suitable surfactant, which may be ionic or non-ionic, for example Triton x100. As a result of cell lysis, the GPI anchored form of CD16b may be anchored to a cell membrane fraction, or released from the cell membrane.

Both the “GPI anchored form of CD16b” and the “intracellular form of CD16b” may be considered to be a form of CD16b that was not actively shed by a neutrophil, or was not actively shed prior to the sample being taken from the subject. In other words, both the “GPI anchored form of CD16b” and the “intracellular form of CD16b” typically have not been cleaved by a protease such as ADAM17 and therefore typically have an intact stalk region. They may also be referred to as “intact” or “non-truncated” or “non-soluble” CD16b. Preferably, at least a substantial proportion of the soluble form of CD16b is removed from the sample. This should be done prior to lysis, so the earlier step of washing the filtrate through the filter membrane with the filtering buffer liquid may be used to achieve this.

The cells may subsequently be lysed whilst in contact with the filter membrane, for example using a lysis buffer (e.g. containing a surfactant) as the processing buffer liquid. Alternatively, the cells may be eluted from the filter, for example into a vessel or onto a device or test strip.

The CD16b level is indicative of the neutrophil level in the sample. In turn, neutrophil levels below a certain threshold are indicative of neutropenia. Subjects with neutropenia are at an increased risk of neutropenic sepsis. Accordingly, the CD16b level is indicative of neutropenia and may be used to determine the risk of neutropenic sepsis or to diagnose neutropenic sepsis, particularly if the subject has one or more symptoms of infection, such as a temperature of above 38° C. Neutrophil levels above a certain threshold are indicative of sepsis.

1. An assay device configured to separate a suspension into a filtrate and a residue for testing at least one of the filtrate and the residue, the device comprising:
a filter aperture for a filter membrane for receiving the suspension and separating the suspension into the filtrate and the residue;
a filtrate receiving portion for receiving the filtrate;
a residue receiving portion for receiving the residue;
a first buffer container configured to contain a first buffer liquid;
a liquid release member configured to create an outlet of the first buffer container;
a filter arm comprising the filter aperture; and
a user actuated switch configured to be movable from a first set position to a second set position, the user actuated switch comprising a transition structure configured to guide the filter arm;
wherein the assay device is configured such that:
at the first set position the filter aperture is aligned with the filtrate receiving portion;
at the second set position the filter aperture is aligned with the first buffer container and with the residue receiving portion; and
during a transition from the first set position to the second set position:
the filter arm is moved such that the filter aperture is aligned with the residue receiving portion; and
the liquid release member and the first buffer container are brought together such that the liquid release member creates the outlet of the first buffer container.

2. The assay device of claim 1 wherein;
the assay device further comprises a second buffer container configured to contain a second buffer liquid;
the user actuated switch is configured to be movable from a third set position to the first set position and from the first set position to the second set position; and
the liquid release member is further configured to create an outlet of the second buffer container.

3. The assay device of claim 2 wherein the assay device is configured such that:
at the third set position the filter aperture is aligned with the filtrate receiving portion;
at the first set position the filter aperture is aligned with the second buffer container and with the filtrate receiving portion; and
during a transition from the third set position to the first set position the liquid release member and the second buffer container are brought together such that the liquid release member creates the outlet of the second buffer container.

4. The assay device of any preceding claim wherein the residue receiving portion is configured to receive the first buffer liquid through the filter aperture.

5. The assay device of claim 4 wherein the residue receiving portion is further configured to receive the residue through the filter aperture.

6. The assay device of any preceding claim wherein the filtrate receiving portion is configured to receive the filtrate through the filter aperture.

7. The assay device of any of claims 2 to 6 wherein the filtrate receiving portion is configured to receive the filtrate and the second buffer liquid through the filter aperture.

8. The assay device of claim 6 or 7 wherein the assay device is configured such that:
at the first set position the filter aperture is aligned with the filtrate receiving portion and with the second buffer container; and
at the second set position the filter aperture is aligned with the residue receiving portion and with the first buffer container.

9. The assay device of any preceding claim wherein the filter aperture contains a filter membrane for receiving the suspension and separating the suspension into the filtrate and the residue.

10. The assay device of any preceding claim wherein the user actuated switch comprises a dial, wherein the dial is configured to be rotated from the first set position to the second set position.

11. The assay device of claim 10 wherein the transition structure is configured to be rotated by rotating the dial.

12. The assay device of any preceding claim wherein the user actuated switch comprises a slider, wherein the slider is configured to be moved in one dimension between the first set position and the second set position.

13. The assay device of any preceding claim wherein the liquid release member comprises a spike.

14. The assay device of any preceding claim wherein the filter arm is configured to rest on the transition structure.

15. The assay device of any preceding claim wherein the transition structure comprises a plurality of levels.

16. The assay device of claim 15 wherein during the transition from the first set position to the second set position, the filter arm is at a different level of the plurality of levels of the transition structure than at the first set position or the second set position.

17. The assay device of any of claims 1 to 16 wherein the filter arm is configured to be rotated by the transition structure.

18. The assay device of claim 17 wherein:
the assay device comprises a first liquid release member configured to create an outlet of the first buffer container
the assay device further comprises a second buffer container configured to contain a second buffer liquid and a second liquid release member configured to create an outlet of the second buffer container; and
the user actuated switch is configured to be movable from a third set position to the first set position and from the first set position to the second set position.

19. The assay device of claim 18 wherein the assay device is configured such that:
at the third set position the filter aperture is aligned with the filtrate receiving portion;
at the first set position the filter aperture is aligned with the second buffer container and with the filtrate receiving portion; and
during a transition from the third set position to the first set position the second liquid release member and the second buffer container are brought together such that the second liquid release member creates the outlet of the second buffer container.

20. The assay device of claim 18 or 19 further comprising a buffer cassette, wherein the buffer cassette is configured to be rotated in response to user actuation of the user actuated switch, and wherein the buffer cassette comprises the first buffer container, the second buffer container, the first liquid release member configured to create an outlet of the first buffer container and the second liquid release member configured to create an outlet of the second buffer container.

21. The assay device of claim 20, wherein the assay device is configured such that during a transition from the third set position to the first set position, the buffer cassette is rotated such that:
the second buffer container is aligned with the filtrate receiving portion; and
the first buffer container is aligned with the residue receiving portion.

22. The assay device of any of claims 18 to 21 wherein each of the first and second buffer containers comprise a first film and a second film, and wherein:
the first liquid release member is configured to pierce the first and second films of the first buffer container; and
the second liquid release member is configured to pierce the first and second films of the second buffer container.

23. The assay device of claim 22, wherein the assay device further comprises a filter membrane in the filter aperture and wherein:
the first liquid release member is configured to pierce the first and second films of the first buffer container and make contact with the filter membrane; and
the second liquid release member is configured to pierce the first and second films of the second buffer container and make contact with the filter membrane.

24. The assay device of claim 23 wherein the first liquid release member and the second liquid release member each comprise a spike that is either smooth, ridged, or serrated.

25. The assay device of any of claims 1 to 16 wherein during a transition from the third set position to the first set position, the filter arm is at a different level of the plurality of levels of the transition structure than at the first set position or the second set position.

26. The assay device of any of claims 1 to 16 and 25 wherein
the assay device further comprises a third buffer container configured to contain a third buffer liquid;
the user actuated switch is configured to be movable from the first set position to the second set position and from the second set position to a fourth set position; and
the liquid release member is further configured to create an outlet of the third buffer container.

27. The assay device of claim 26 wherein the assay device is configured such that:
at the fourth set position the filter aperture is aligned with the residue receiving portion and with the third buffer container;
during a transition from the second set position to the fourth set position the liquid release member and the third buffer container are brought together such that the liquid release member creates the outlet of the third buffer container.

28. The assay device of any of claims 1 to 16 and 25 wherein:
the assay device further comprises a second buffer container configured to contain a second buffer liquid and a third buffer container configured to contain a third buffer liquid;
the user actuated switch is configured to be movable from a third set position to the first set position, from the first set position to the second set position, and from the second set position to a fourth set position; and
the liquid release member is further configured to create an outlet of the second buffer container and to create an outlet of the third buffer container.

29. The assay device of claim 28 wherein the assay device is configured such that:
at the third set position the filter aperture is aligned with the filtrate receiving portion;
at the first set position the filter aperture is aligned with the second buffer container and with the filtrate receiving portion;
at the fourth set position the filter aperture is aligned with the residue receiving portion and with the third buffer container;
during a transition from the third set position to the first set position the liquid release member and the second buffer container are brought together such that the liquid release member creates the outlet of the second buffer container; and
during a transition from the second set position to the fourth set position the liquid release member and the third buffer container are brought together such that the liquid release member creates the outlet of the third buffer container.

30. The assay device of any of claims 14, 15 and 24 to 28 wherein the plurality of levels are connected via a plurality of ramps.

31. The assay device of any of claims 14, 15 and 24 to 29 wherein the transition structure is configured to guide the filter arm along the plurality of levels and between the plurality of levels in response to movement of the user actuated switch from the first set position to the second set position.

32. The assay device of any of claims 15, 16 and 25 to 29 wherein the plurality of levels comprise concentric arcs.

33. The assay device of claim 32 wherein at least one of the plurality of levels comprises more than one arc.

34. The assay device of any preceding claim wherein the filter arm further comprises a first contact point configured to rest on the transition structure.

35. The assay device of any of claims 1 to 16 or 25 to 34 further comprising a sample receiving arm, the sample receiving arm comprising the filtrate receiving portion and the residue receiving portion.

36. The assay device of claim 35 wherein the transition structure is configured to guide the sample receiving arm in response to movement of the user actuated switch.

37. The assay device of claim 36 wherein the sample receiving arm further comprises a contact aperture and a second contact point configured to rest on the transition structure, wherein the filter arm rests on the sample receiving arm and wherein the first contact point is configured to pass through the contact aperture and rest on the transition structure.

38. The assay device of any of claims 15, 16 and 25 to 37 wherein the plurality of levels comprise a first level, a second level below the first level and a third level below the second level.

39. The assay device of claim 38 wherein the sample receiving arm comprises a second contact point and the filter arm comprises a first contact point.

40. The assay device of claim 39 wherein the plurality of levels are configured such that during the transition from the third set position to the first set position the first contact point moves from the third level to the second level, and the second contact moves from the second level to the first level.

41. The device of claim 39 or 40 wherein the plurality of levels are configured such that during the transition from the third set position to the first set position:
i) the first contact point moves from the second level to the third level and the second contact point moves from the first level to the second level; and
ii) the first contact point moves from the third level to the second level, and the second contact moves from the second level to the first level.

42. The assay device of any of claims 39 to 41 wherein the plurality of levels are configured such that during the transition from the first set position to the second set position:
i) the first contact point moves from the second level to the third level, and the second contact point moves from the first level to the second level; and
ii) the first contact point moves from the third level to the second level and the second contact point remains at the second level, such that the upper arm is lifted relative to the lower arm and the upper arm rotates relative to the lower arm such that the filter aperture is aligned with the residue receiving portion.

43. The assay device of any of claims 39 to 42 wherein the plurality of levels are configured such that during the transition from the second set position to the fourth set position:
i) the first contact point moves from the second level to the third level and the second contact point remains at the second level; and
ii) the first contact point moves from the third level to the second level and the second contact point moves from the second level to the first level.

44. The assay device of claim 41 wherein the plurality of levels are configured such that during the transition from the third set position to the first set position the liquid release member creates the outlet of the second buffer container when the first contact point moves from the third level to the second level.

45. The assay device of claim 42 wherein the plurality of levels are configured such that during the transition from the first set position to the second set position the liquid release member creates the outlet of the first buffer container when the first contact point moves from the third level to the second level.

46. The assay device of claim 43 wherein the plurality of levels are configured such that during the transition from the second set position to the fourth set position the liquid release member creates the outlet of the third buffer container when the first contact point moves from the third level to the second level.

47. The device of any of claims 2 to 16 and 25 to 46 wherein the transition structure is configured such that during the transition from the third set position to the first set position the liquid release member is configured to move along the second buffer container after it has created the outlet of the second buffer container.

48. The device of any of claims 1 to 16 and 25 to 47 wherein the transition structure is configured such that during the transition from the first set position to the second set position the liquid release member is configured to move along the first buffer container after it has created the outlet of the first buffer container.

49. The device of any of claims 26 to 48 wherein the transition structure is configured such that during the transition from the second set position to the first set position the liquid release member is configured to move along the third buffer container after it has created the outlet of the third buffer container.

50. The device of any of claims 1 to 16 and 25 to 49 wherein the filter arm comprises the liquid release member.

51. The assay device of any preceding claim wherein the filtrate receiving portion comprises a lateral flow test strip.

52. The assay device of any of claims 1 to 50 wherein the filtrate receiving portion comprises a lateral flow dummy strip.

53. The assay device of claim any preceding claim wherein the residue receiving portion comprises a lateral flow test strip.

54. The assay device of any of claims 1 to 52 wherein the residue receiving portion comprises a microfluidic channel.

55. The assay device of claim 51 wherein the filtrate receiving portion is configured to test for a sepsis marker.

56. The assay device of claim 53 or 54 wherein the residue receiving portion is configured to test for CD16b.
